(19) United States
(12) Patent Application Publication
(10) Pub. No.: US 2024/0342065 A1
(43) Pub. Date: Oct. 17, 2024

(54) PERSONAL CARE COMPOSITIONS AND METHODS COMPRISING TRANS-1-CHLORO-3,3,3-TRIFLUOROPROPENE AND TRANS-1,3,3,3-TETRAFLUOROPROPENE

(71) Applicant: HONEYWELL INTERNATIONAL INC., Charlotte, NC (US)

(72) Inventors: CHANGMING WANG, Shenzhen (CN); YIU KEUNG LING, Charlotte, NC (US); BARRY SETIAWAN, Charlotte, NC (US)

(73) Assignee: HONEYWELL INTERNATIONAL INC., Charlotte, NC (US)

(21) Appl. No.: 18/656,468

(22) Filed: May 6, 2024

Related U.S. Application Data
(63) Continuation of application No. 17/061,545, filed on Oct. 1, 2020, now abandoned.
Application No. 17/061,545 is a continuation-in-part of application No. PCT/CN2020/082758, filed on Apr. 1, 2020.
Application No. PCT/CN2020/082758 is a continuation-in-part of application No. PCT/CN2020/080355, filed on Mar. 20, 2020.

(51) Int. Cl. A61K 8/31; A45D 34/00; A61K 8/04; A61K 8/06; A61Q 5/00; A61Q 19/00

(52) U.S. Cl. CPC A61K 8/315; A45D 34/00; A61K 8/046; A61K 8/062; A61Q 5/00; A61Q 19/00; A61K 2800/48

(57) ABSTRACT

A method of treating human skin, hair and/or mucous membranes using a personal care composition in the form of a stable oil-in-water emulsion comprising: (i) a continuous aqueous phase comprising water; (ii) a discontinuous oil phase comprising trans-1-chloro-3,3,3-trifluoropropene and/or trans-1-3,3,3-tetrafluoropropene and when present together are present in a trans1233zd:trans1234ze weight ratio of from about 1:18 to less than about 8:1; and (iii) an emulsifier, which may be present in the continuous and/or discontinuous phase, comprising (a) a non-ionic emulsifier having an HLB of from about 7 to about 19; and/or (b) a polymeric emulsifier.

### CROSS REFERENCE TO RELATED APPLICATIONS

The present application is a continuation of U.S. application Ser. No. 17/061,545, filed Oct. 1, 2020, which is a continuation-in-part of PCT Application No. PCT/CN2020/082758, filed Apr. 1, 2020. The present application is also a continuation-in-part of PCT Application No. PCT/CN2020/080355, filed Mar. 20, 2020, each of which is incorporated herein by reference in its entirety.

### FIELD OF THE INVENTION

The present invention relates to personal care compositions and to methods and devices for providing personal care, including but not limited to compositions, articles and methods that provide novel foam forms and/or which provide novel foaming effects.

### BACKGROUND

Personal care compositions, such as skin lotions and cleansers and shaving creams (including foams and gels), generally comprise several components in addition to the active ingredient. Such additional ingredients may include, for example, thickeners, suspending agents, surfactants and foaming agents, which are included in order to effect a property of the composition as it is being dispensed and/or upon application to the skin.

One particular type of formulation is a gel which is self-foaming either upon being dispensed and/or upon being rubbed into or onto the area of the skin or scalp being treated. For example, one of the more popular forms of personal care products used today is the post-foaming shave gel. In such post-foaming compositions, it is common to add soaps, surfactants, active ingredients and other components with relatively volatile hydrocarbons in an effort to form clear, stable gels or emulsions when kept under pressure. Once these materials are dispensed and mechanically agitated, the gels form foams, generally due to the post-dispensing addition of energy to the gel.

Applicants have come to appreciate that the performance of prior personal care formulations, including post-foaming and non post-foaming formulations, is lacking and/or could be improved in several respects. By way of example, applicants have found that in many prior formulations the volatile hydrocarbons that were heretofore used to form the foam in post-foam applications resulted in the degradation of other desirable aspects of the formulation. For example, applicants have come to appreciate that such prior post-foam formulations produced a foam too quickly after application, which tends to negate the ability of the formulation to spread on the skin or hair of the user, and/or to penetrate into the skin/hair to the extent that would be desirable to the user. Furthermore, even to the relatively small extent that prior post-foam formulations were able to spread on the skin prior to foaming, such formulations did not enhance or improve the ability of the active ingredient to penetrate into the pores of the skin and thereby provide both the perception and the actuality of improved performance.

Furthermore, applicants have come appreciate that in many prior formulations the look and/or initial sensation on the skin/scalp is not as attractive to the user as could be achieved and detracts substantially from the satisfaction of the user when the product is used for its intended purpose. For example, applicants have come appreciate that if a foam or gel that is otherwise acceptable for its intended purpose but appears when dispensed to drippy, soggy and/or otherwise unattractive (such as might occur if the material contained in and/or dispensed from be container is opaque, hazy or fogy), the success of the product could be unacceptable or at least less than desired.

One method which can be used to improve the transparency of an emulsion formulation, and hence reduce opacity, has been to use a microelmulsion in which the discontinuous droplet size of the emulsion is less than about 25% of the wavelength of light. However, such techniques can be disadvantageous because they require relatively high surfactant concentrations, high levels of mixing energy to form the emulsion, and/or may cause skin irritation when used in a personal care treatment formulation.

Another aspect of prior formulations has been the ability to provide the user with the sensation of cooling as a result of application of the personal care product to the skin and/or hair. Such sensation has been provided in many prior formulations by the inclusion of certain ingredients known as sensates, which manipulate the transient receptor potential (TRP) channels contained in human tissue. See, for example, US 2017/0000713. Applicants have come to appreciate that the use of such sensate ingredients can have several disadvantages, including relatively high cost of these ingredients and their derivatives. Another potential disadvantage is the need for sensates to interact directly with the biological function of the user, which can be undesirable in certain cases/applications, such as users who may be especially sensitive to and/or allergic to the direct biological interaction and who might experience longer-lasting side effects as a result of the ingredient remaining in contact with the user. Accordingly, applicants have come to appreciate the desirability of achieving such a cooling sensation using a simpler and less costly mechanism and without the above-noted other disadvantages of sensates. Furthermore, applicants have come to appreciate the desire of many users to experience the cooling sensation that appears essentially immediately upon application to the skin/hair/mucous membrane, and these sensates have limited use in those skin, body and mucous membrane applications where an apparently instant refreshing cooling sensation is desired. Moreover, applicants have also come to appreciate the advantage of treatment compositions and methods in which the cooling effect is long lasting and preferably at the same time also being perceived to provide essentially immediate cooling sensation upon application.

Applicants have also come to appreciate that the use of certain materials, as described hereinafter, to form the foam can have advantages in the production of a desirable and/or novel foam forms. However, applicants have also come to appreciate that in many such cases the ability to maintain the components in a stable form while in the container under pressure can be a substantial challenge with non-predictable results.

### BRIEF DESCRIPTION OF THE DRAWING

The patent or application file contains at least one drawing executed in color. Copies of this patent or patent application publication with color drawing(s) will be provided by the Office upon request and payment of the necessary fee.

FIGS. 1A and 1B are color photographs showing ice cream foams produced according to an embodiment of the present invention as described in Example 1.

FIG. 2 is a color photograph showing ice cream foams produced according to an embodiment of the present invention as described in Example 3.

FIG. 3 is a color photograph showing ice cream foams produced according to an embodiment of the present invention as well as foams that are not ice cream foam as described in Example 4.

FIG. 4 is a color photograph showing crackling sherbet foams produced according to the experiment described in Example 5.

FIG. 5 is a color photograph showing crackling sherbet foams produced according to the experiment described in Example 6.

FIG. 6 is a color photograph showing a crackling sherbet foam produced according to the experiment described in Example 7.

FIG. 7 is a color photograph showing crackling sherbet foams produced according to the experiment described in Example 8.

FIG. 8 is a color photograph showing crackling sherbet foams produced according to the experiment described in Example 9.

FIG. 9 is a color photograph showing a crackling sherbet foam produced according to the experiment described in Example 10.

FIG. 10 is a color photograph showing sherbet, crackling foams according to an embodiment of the present invention as well as foams that are not sherbet foam, as described in Example 11.

FIGS. 11-14 are color photographs showing clear personal care formulations as described in Example 13.

FIGS. 15 and 16 are color photographs showing clear personal care formulations as described in Example 14.

FIG. 17 is a color photograph showing a semi-transparent personal care formulation as described in Example 15.

FIGS. 18A and 18B are color photographs showing clear personal care formulation as described in Example 16.

FIG. 19 is a color photograph showing a personal care foam as described in Example 18.

FIG. 20 is a color photograph showing a personal care foam as described in Example 21.

### SUMMARY

Applicants have come to appreciate that one or more of the disadvantages and failures described above in connection with prior formulations and methods, and preferably all of such disadvantages and failures, can be reduced or substantially eliminated using the methods, articles and personal care formulations according to the present invention. In addition and/or in the alternative, applicants have developed highly desirable and/or novel formulations and foam forms that were not heretofore known or available.

The present invention includes methods of treating human skin, hair and/or mucous membranes comprising:
(a) providing a personal care composition in a container under pressure in the form of a stable oil-in-water emulsion comprising:
   (i) a continuous aqueous phase comprising water;
   (ii) a discontinuous oil phase phase comprising trans-1-chloro-3,3,3-trifluoropropene (hereinafter 1233zd(E)) and/or trans-1-3,3,3-tetrafluoropropene (hereinafter 1234ze(E)), wherein said 1233zd(E) and said 1234ze (E) together comprise at least about 40% by weight of said personal care composition and are present in a trans1233zd:trans1234ze weight ratio of from about 1:18 to less than about 8:1; and
   (iii) an emulsifier, which may be present in the continuous and/or discontinuous phase, comprising (a) a non-ionic emulsifier having an HLB of from about 7 to about 18; and/or (b) a polymeric emulsifier, preferably an emulsifying acrylate-based copolymer; (c) and optionally a cationic emulsifier, said emulsifier (iii) being present in an amount effective to ensure that said oil-in-water emulsion is stable, and; v) one or more active ingredients which may be present in the continuous and/or discontinuous phase; and
   (vi) optionally one or more auxiliary gel, lotion or cream forming components which may be present in the continuous and/or discontinuous phase; and
(b) dispensing said personal care composition on and/or into the skin, hair and/or mucous membrane of the human being treated by discharging at least a portion of the personal care composition from said container into ambient pressure conditions to form a material on the user in a form selected from sherbet foam; ice cream foam; crackling foam; ultra crackling foam, bubbling foam; clear foam, gel and/or lotion; or a combination of two or more of these, with the provisos that:
   (1) for said sherbet foam and said crackling foam the weight ratio of said 1233zd to said 1234ze is from 8:1 to about 1:1.5, said emulsifier comprises said non-ionic emulsifier and said personal care composition comprises a thickener for said continuous and/or said discontinuous phase;
   (2) for said ice cream foam (i) the weight ratio of said 1233zd to said 1234ze is from 1:1.5 to about 1:8; (ii) said emulsifier comprises a solid non-ionic emulsifier having an HLB of from about 7 to about 19 and wherein the net HLB of the emulsifiers in formulation is from about 7 to 19; (iii) said personal care composition comprises a solid oil phase thickener (preferably fatty acid alcohol and/or cationic emulsifier and/or a high melting point wax) and optionally an aqueous phase thickener, provided that the weight ratio of the continuous phase thickener to the discontinuous phase thickener is equal to or less than 0.02, or less than about 0.015, or less than 0.01, or less than 0.005; (iv) and said personal care composition comprises at least about 3% by weight of oil phase liquids, wherein the weight ratio of said oil phase thickeners to said total oil phase liquids is greater than 1.25 and less than about 3 and;
   (3) for said ultra crackling foam, the personal care composition comprises at least about 60% by weight of said 1234ze, said ratio of said 1233zd to said 1234ze is from about 1:4 to about 1:18, said emulsifier comprises said non-ionic liquid emulsifier, and greater than 3% by weight and less than about 7% by weight based on all to components in the personal care composition of one or more polar carriers having a normal boiling point or an initial boiling point of from about 60° C. to about 85° C., preferably comprising, consisting essentially of or consisting of ethanol;
   (4) for bubbling foam, the weight ratio of said 1233zd to said 1234ze is from 8:1 to about 1:1.5 and said emulsifier comprises said polymeric emulsifier in a weight ratio of total oil components to polymeric emulsifier of from about greater than about 515 and less than about 740; and
   (5) for clear foam, gel and/or lotion, said discontinuous phase comprises a discontinuous phase refractive index balancer (DRIB) in an amount effective to produce a weight ratio of said 1233zd(E):DRIB of from about 3:1 to about 30:1. For the purposes of convenience, a method according to the present invention is referred to herein as Method 1.

As used herein, the term “ambient pressure conditions” means at or about atmospheric pressure.

As used herein, the term “under pressure” means at a pressure above ambient pressure conditions.

As used herein, the term “discontinuous phase refractive index balancer” (DRIB) means a component or combination of components that are soluble in said discontinuous phase, have a refractive index equal to or greater than 1.35, and do not interfere with or substantially reduce the stability of the emulsion.

As used herein, the term “continuous phase refractive index balancer” (CRIB) means a component or combination of components that are soluble in said continuous phase, have a refractive index equal to or greater than about 1.375, and do not interfere with or substantially reduce the stability of the emulsion.

As used herein, the term “sherbet foam” means a foam that is translucent and has a textured surface. A photograph exemplary/representative of a sherbet foam is provided in connection with the description thereof in the examples.

As used herein, the term “ice cream foam” means a foam that has a highly textured, non-smooth surface that contains numerous relatively sharp but soft points/discontinuities. A photograph exemplary/representative of a ice cream foam is provided in connection with the description thereof in the examples.

As used herein, the term “crackling foam” means a foam that produces a subtle crackling sound after it has been applied.

As used herein, the term “ultra-crackling foam” means a foam that produces a crackling sound that is loud and distinct after it has been applied.

As used herein, the term “bubbling foam” means a foam that has bubbling activity on its surface after it has been applied.

As used herein, the term “clear foam, gel or lotion” is a foam, gel or lotion, respectively, that is essentially transparent.

As used herein, the term “stable oil-in-water emulsion” means an emulsion that forms upon mixing and does not separate into visibly distinct phases upon standing for a period of at least about 24 hours at about room temperature.

As used herein, the term “solid non-ionic emulsifier” means a non-ionic emulsifier that has a melting point above about room temperature.

As used herein, the term “liquid non-ionic emulsifier” means a non-ionic emulsifier that has a melting point about below room temperature.

As used herein, the term “normal” with respect to boiling point or melting point means the indicated value measure at about 1 atmosphere.

As used herein, the term “net HLB” means the average HLB of the emulsifiers in a compositions calculated on the relative weight fraction of all of the identified emulsifiers in the composition.

As used herein, the term “solid discontinuous phase thickener” means the identified thickener has a melting point above room temperature and which is miscible in the discontinuous phase.

As used herein, the term “oil phase liquids” means the identified material has a melting point below room temperature and which is miscible in the oil phase or discontinuous phase.

As used herein, the term “silicone oil” means silicone component(s) that are useful in personal care formulations and which have a melting point below room temperature.

As used herein, the term “all components in the discontinuous phase” means all the components in the oil phase in an oil-in-water emulsion and is intended to include each of the emulsifier components and the 1233zd(e) and 1234ze(E), unless specifically indicated herein.

The present invention includes methods of treating human skin, hair and/or mucous membranes using an ice cream foam comprising:
(a) providing a personal care composition in a container under pressure in the form of a stable oil-in-water emulsion comprising:
   (i) a continuous aqueous phase comprising water;
   (ii) a discontinuous oil phase comprising 1233zd(E)) and 1234ze(E), wherein said 1233zd(E) and said 1234ze(E) together comprise at least about 40% by weight of said personal care composition and are present in a trans1233zd:trans1234ze weight ratio of from about 1:1.5 to 1:8; and
   (iii) an emulsifier, which may be present in the continuous and/or discontinuous phase, comprising (a) one or more solid non-ionic emulsifiers having an HLB of from about 7 to about 19 wherein the net HLB of the formulation is from about 7 to about 19; (b) optionally a polymeric emulsifier, preferably an emulsifying acrylate-based copolymer; and (c) optionally a cationic emulsifier, said emulsifier (iii) being present in an amount effective to ensure that said oil-in-water emulsion is stable;
   (iv) a solid thickener for said discontinuous oil phase, preferably fatty acid alcohol and/or cationic emulsifier and/or a high melting point wax, and optionally an aqueous phase thickener, provided that the weight ratio of the aqueous phase thickener to the oil phase thickener is equal to or less than 0.02, or less than about 0.015, or less than 0.01, or less than 0.005; and
   (v) at least about 3% by weight of oil phase liquids, wherein the weight ratio of said oil phase thickeners to said total oil phase liquids is greater than 1.25 and less than about 3 and wherein oil phase liquid comprises at least one silicone oil; and
   (vi) optionally one or more auxiliary gel, lotion or cream forming components which may be present in the continuous and/or discontinuous phase; and
(b) dispensing said personal care composition on and/or into the skin, hair and/or mucous membrane of the human being treated by discharging at least a portion of the personal care composition from said container into ambient pressure conditions to form a material on the user in the form of ice cream foam. For the purposes of convenience, a method according to the present invention is referred to herein as Method 2.

The present invention includes methods of treating human skin, hair and/or mucous membranes comprising using a sherbet foam:
(a) providing a personal care composition in a container under pressure in the form of a stable oil-in-water emulsion comprising:
   (i) a continuous aqueous phase comprising water;
   (ii) a discontinuous oil phase comprising 1233zd(E) and 1234ze(E), wherein said 1233zd(E) and said 1234ze (E) together comprise at least about 40% by weight of said personal care composition and are present in a trans1233zd:trans1234ze weight ratio of from about 8:1 to about 1:1.5; and
   (iii) one or more emulsifiers, which may be present in the continuous and/or discontinuous phase, having a net HLB of from about 7 to about 19 and comprising at least one non-ionic emulsifier; (b) optionally a polymeric emulsifier, preferably an emulsifying acrylate-based copolymer; and (c) optionally a cationic emulsifier, said emulsifier (iii) being present in an amount effective to ensure that said oil-in-water emulsion is stable, and;
   (iv) a thickener for said continuous and/or said discontinuous phase;
   (iv) one or more active ingredients which may be present in the continuous and/or discontinuous phase; and
   (v) optionally one or more auxiliary gel, lotion or cream forming components which may be present in the continuous and/or discontinuous phase; and (b) dispensing said personal care composition on and/or into the skin, hair and/or mucous membrane of the human being treated by discharging at least a portion of the personal care composition from said container into ambient pressure conditions to form a material on the user in the form of a sherbet foam. For the purposes of convenience, a method according to the present invention is referred to herein as Method 3.

The present invention includes methods of treating human skin, hair and/or mucous membranes using an ultra-crackling foam comprising:
(a) providing a personal care composition in a container under pressure in the form of a stable oil-in-water emulsion comprising:
(i) a continuous aqueous phase comprising water and greater than 3% by weight and less than about 7% by weight based on all to components in the personal care composition of one or more polar carriers having a normal boiling point or an initial boiling point of from about 60° C. to about 85° C., preferably comprising, consisting essentially of or consisting of ethanol;
(ii) a discontinuous phase comprising 1233zd(E) and 1234ze(E)) wherein said 1233zd(E) and said 1234ze (E) together comprise at least about 60% by weight of said personal care composition and are present in a trans1233zd:trans1234ze weight ratio of from about 1:18 to less than about 1:4; and
(iii) an emulsifier, which may be present in the continuous and/or discontinuous phase, comprising (a) one or more liquid non-ionic emulsifiers having an HLB of from about 7 to about 19 wherein the net HLB of the formulation is from about 7 to about 19 comprising; (b) optionally a polymeric emulsifier, preferably an emulsifying acrylate-based copolymer; (c) and optionally a cationic emulsifier, said emulsifier (iii) being present in an amount effective to ensure that said oil-in-water emulsion is stable;
(iv) one or more active ingredients which may be present in the continuous and/or discontinuous phase; and
(v) optionally one or more auxiliary gel, lotion or cream forming components which may be present in the continuous and/or discontinuous phase; and
(b) dispensing said personal care composition on and/or into the skin, hair and/or mucous membrane of the human being treated by discharging at least a portion of the personal care composition from said container into ambient pressure conditions to form a material on the user in the form of an ultra crackling foam. For the purposes of convenience, a method according to the present invention is referred to herein as Method 4.

The present invention includes methods of treating human skin, hair and/or mucous membranes using a bubbling foam comprising:
(a) providing a personal care composition in a container under pressure in the form of a stable oil-in-water emulsion comprising:
(i) a continuous aqueous phase comprising water;
(ii) a discontinuous oil phase comprising 1233zd(E) and 1234ze(E), wherein said 1233zd(E) and said 1234ze (E) together comprise at least about 40% by weight of said personal care composition and are present in a trans1233zd:trans1234ze weight ratio of from about 4:1 to about 1:1; and
(iii) an emulsifier, which may be present in the continuous and/or discontinuous phase, comprising a polymeric emulsifier, preferably an emulsifying acrylate-based copolymer; optionally (b) a cationic emulsifier and/or non-ionic emulsifier, said emulsifier (iii) being present in an amount effective to ensure that said oil-in-water emulsion is stable and wherein the weight ratio of all the components in the discontinuous phase, preferably the oil phase, to said polymeric emulsifier is from about greater than about 515 and less than about 740, and;
(iv) one or more active ingredients which may be present in the continuous and/or discontinuous phase; and
(v) optionally one or more auxiliary gel, lotion or cream forming components which may be present in the continuous and/or discontinuous phase; and
(b) dispensing said personal care composition on and/or into the skin, hair and/or mucous membrane of the human being treated by discharging at least a portion of the personal care composition from said container into ambient pressure conditions to form a material on the user in the form of bubbling foam. For the purposes of convenience, a method according to the present invention is referred to herein as Method 5.

The present invention includes methods of treating human skin, hair and/or mucous membranes using clear foam, gel and/or lotion comprising:
(a) providing a personal care composition in a container in the form of a stable oil-in-water emulsion comprising:
(i) a continuous aqueous phase comprising water;
(ii) a discontinuous phase comprising trans-1-chloro-3,3,3-trifluoropropene (hereinafter 1233zd(E)), wherein said discontinuous phase comprises a discontinuous phase refractive index balancer (DRIB) in an amount effective to produce a weight ratio of said 1233zd(E):DRIB of from about 3:1 to about 30:1;
(iii) one or more active ingredients which may be present in the continuous and/or discontinuous phase; and
(iv) optionally one or more auxiliary gel, lotion or cream forming components which may be present in the continuous and/or discontinuous phase; and
(b) dispensing said personal care composition on and/or into the skin, hair and/or mucous membrane of the human being treated by discharging at least a portion of the personal care composition from said container into ambient pressure conditions to form a material on the user in a form of a clear foam, gel and/or lotion; or a combination of two or more of these. For the purposes of convenience, a method according to the present invention is referred to herein as Method 6.

The present invention includes a personal care formulation for treating human skin, hair and/or mucous membranes to form an ice cream foam comprising a stable oil-in-water emulsion comprising:
(i) a continuous aqueous phase comprising water;
(ii) a discontinuous oil phase comprising 1233zd(E)) and 1234ze(E), wherein said 1233zd(E) and said 1234ze(E) together comprise at least about 40% by weight of said personal care composition and are present in a trans1233zd:trans1234ze weight ratio of from about 1:1.5 to 1:8; and
(iii) an emulsifier, which may be present in the continuous and/or discontinuous phase, comprising (a) one or more solid non-ionic emulsifiers having an HLB of from about 7 to about 19 wherein the net HLB of the formulation is from about 7 to about 19; (b) optionally a polymeric emulsifier, preferably an emulsifying acrylate-based copolymer; and (c) optionally a cationic emulsifier, said emulsifier (iii) being present in an amount effective to ensure that said oil-in-water emulsion is stable;
(iv) a solid thickener for said discontinuous oil phase, preferably fatty acid alcohol and/or cationic emulsifier and/or a high melting point wax, and optionally an aqueous phase thickener, provided that the weight ratio of the aqueous phase thickener to the oil phase thickener is equal to or less than 0.02, or less than about 0.015, or less than 0.01, or less than 0.005;
(v) at least about 3% by weight of oil phase liquids, wherein the weight ratio of said oil phase thickeners to said total oil phase liquids is greater than 1.25 and less than about 3 and wherein oil phase liquid comprises at least one silicone oil; and
(vi) optionally one or more auxiliary gel, lotion or cream forming components which may be present in the continuous and/or discontinuous phase. For the purposes of convenience, a personal care composition according to the present invention is referred to herein as Composition 1.

The present invention includes a personal care formulation for treating human skin, hair and/or mucous membranes to form an sherbet foam in the form of a stable oil-in-water emulsion comprising:
(i) a continuous aqueous phase comprising water;
(ii) a discontinuous oil phase comprising 1233zd(E) and 1234ze(E), wherein said 1233zd(E) and said 1234ze (E) together comprise at least about 40% by weight of said personal care composition and are present in a trans1233zd:trans1234ze weight ratio of from about 8:1 to about 1:1.5; and
(iii) one or more emulsifiers, which may be present in the continuous and/or discontinuous phase, having a net HLB of from about 7 to about 19 and comprising at least one non-ionic emulsifier; (b) optionally a polymeric emulsifier, preferably an emulsifying acrylate-based copolymer; and (c) optionally a cationic emulsifier, said emulsifier (iii) being present in an amount effective to ensure that said oil-in-water emulsion is stable, and;
(iv) a thickener for said continuous and/or said discontinuous phase;
(iv) one or more active ingredients which may be present in the continuous and/or discontinuous phase; and
(v) optionally one or more auxiliary gel, lotion or cream forming components which may be present in the continuous and/or discontinuous phase. For the purposes of convenience, a personal care composition according to the present invention is referred to herein as Composition 2.

The present invention includes a personal care formulation for treating human skin, hair and/or mucous membranes to form an ultra-crackling foam in the form of a stable oil-in-water emulsion comprising:
(i) a continuous aqueous phase comprising water and greater than 3% by weight and less than about 7% by weight based on all to components in the personal care composition of one or more polar carriers having a normal boiling point or an initial boiling point of from about 60° C. to about 200° C., preferably comprising, consisting essentially of or consisting of ethanol;
(ii) a discontinuous phase comprising 1233zd(E) and 1234ze(E)) wherein said 1233zd(E) and said 1234ze (E) together comprise at least about 60% by weight of said personal care composition and are present in a trans1233zd:trans1234ze weight ratio of from about 1:18 to less than about 1:4; and
(iii) an emulsifier, which may be present in the continuous and/or discontinuous phase, comprising (a) one or more liquid non-ionic emulsifiers having an HLB of from about 7 to about 19 wherein the net HLB of the formulation is from about 7 to about 19 comprising; (b) optionally a polymeric emulsifier, preferably an emulsifying acrylate-based copolymer; (c) and optionally a cationic emulsifier, said emulsifier (iii) being present in an amount effective to ensure that said oil-in-water emulsion is stable;
(iv) one or more active ingredients which may be present in the continuous and/or discontinuous phase; and
(v) optionally one or more auxiliary gel, lotion or cream forming components which may be present in the continuous and/or discontinuous phase. For the purposes of convenience, a personal care composition according to the present invention is referred to herein as Composition 3.

The present invention includes a personal care formulation for treating human skin, hair and/or mucous membranes to form an bubbling foam in the form of a stable oil-in-water emulsion comprising:
(i) a continuous aqueous phase comprising water;
(ii) a discontinuous oil phase comprising 1233zd(E) and 1234ze(E), wherein said 1233zd(E) and said 1234ze (E) together comprise at least about 40% by weight of said personal care composition and are present in a trans1233zd:trans1234ze weight ratio of from about 8:1 to about 1:1; and
(iii) an emulsifier, which may be present in the continuous and/or discontinuous phase, comprising a polymeric emulsifier, preferably an emulsifying acrylate-based copolymer; optionally (b) a cationic emulsifier and/or non-ionic emulsifier, said emulsifier (iii) being present in an amount effective to ensure that said oil-in-water emulsion is stable and wherein the weight ratio of all the components in the discontinuous phase, preferably the oil phase, to said polymeric emulsifier is from about greater than about 515 and less than about 740, and;
(iv) one or more active ingredients which may be present in the continuous and/or discontinuous phase; and
(v) optionally one or more auxiliary gel, lotion or cream forming components which may be present in the continuous and/or discontinuous phase. For the purposes of convenience, a personal care composition according to the present invention is referred to herein as Composition 4.

The present invention includes a personal care formulation for treating human skin, hair and/or mucous membranes to form an clear foam, gel and/or lotion in the form of a stable oil-in-water emulsion comprising:
(i) a continuous aqueous phase comprising water;
(ii) a discontinuous phase comprising trans-1-chloro-3,3,3-trifluoropropene (hereinafter 1233zd(E)), wherein said discontinuous phase comprises a discontinuous phase refractive index balancer (DRIB) in an amount effective to produce a weight ratio of said 1233zd(E):DRIB of from about 3:1 to about 30:1;
(iii) one or more active ingredients which may be present in the continuous and/or discontinuous phase; and
(iv) optionally one or more auxiliary gel, lotion or cream forming components which may be present in the continuous and/or discontinuous phase. For the purposes of convenience, a personal care composition according to the present invention is referred to herein as Composition 5.

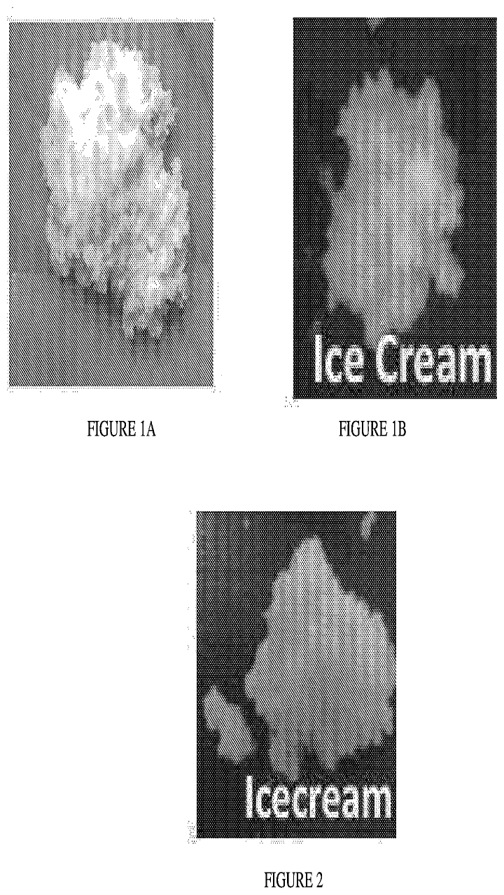

The present invention also includes an ice cream foam, preferably having the appearance and texture as illustrated in FIGS. 1A, 1B and 2 hereof. For the purposes of convenience, a personal care composition according to the present invention is referred to herein as Ice Cream Foam 1.

The present invention also includes an ice cream foam made in accordance with Method 2. For the purposes of convenience, a personal care composition according to the present invention is referred to herein as Ice Cream Foam 2.

For the purposes of clarity, when the phrase “in accordance with Method” of any designated number (such as “in accordance with Method 2” in the preceding paragraph) is used, it is intended to refer to all of the definitions of the numbered Method, including any such numbered Method have a suffix. This, by way of example, the reference herein to “in accordance with Method 2” is intended to mean each method within the definition of Method 2, including any suffixed version of Method 2, such as Method 2A, Method 2B, Method 2C, and so on that are defined herein.

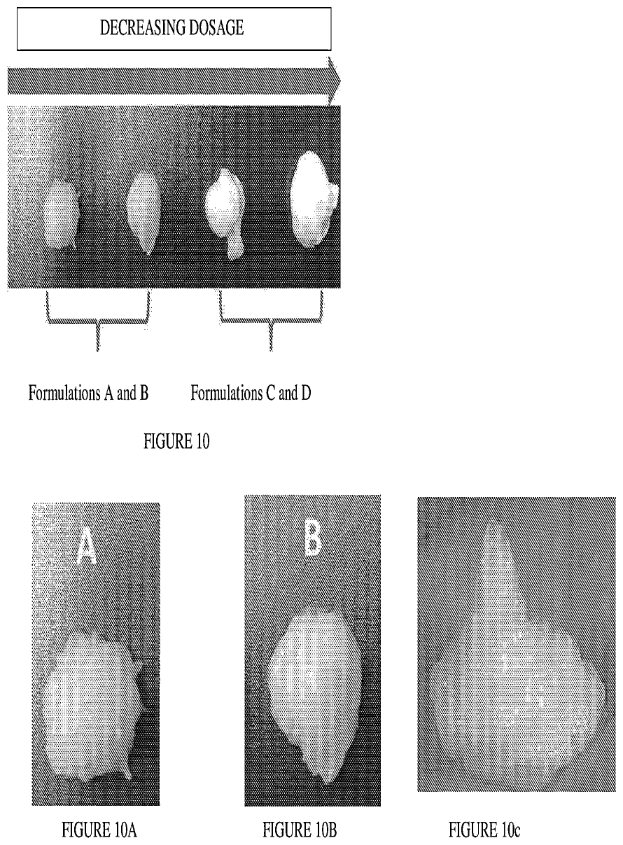

The present invention also includes a sherbet foam, preferably having the appearance and texture as illustrated in FIGS. 10A, 10B and 10C hereof. For the purposes of convenience, a personal care composition according to the present invention is referred to herein as Sherbet Foam 1.

The present invention also includes an sherbet foam made in accordance with Method 3. For the purposes of convenience, a personal care composition according to the present invention is referred to herein as Sherbet Foam 2.

The present invention also includes an ultra-crackling foam. For the purposes of convenience, a personal care composition according to the present invention is referred to herein as Ultra-crackling Foam 1.

The present invention also includes an ultra-crackling foam made in accordance with Method 4. For the purposes of convenience, a personal care composition according to the present invention is referred to herein as Ultra-crackling Foam 2.

The present invention also includes a bubbling foam. For the purposes of convenience, a personal care composition according to the present invention is referred to herein as Bubbling Foam 1.

The present invention also includes a bubbling foam made in accordance with Method 5. For the purposes of convenience, a personal care composition according to the present invention is referred to herein as Bubbling Foam 2.

The present invention also includes a clear foam, lotion or gel. For the purposes of convenience, a personal care composition according to the present invention is referred to herein as Clear Foam 1.

The present invention also includes a clear foam, lotion or gel made in accordance with Method 6. For the purposes of convenience, a personal care composition according to the present invention is referred to herein as Clear Foam 2.

### DETAILED DESCRIPTION

The preferred methods, foams, gels, lotions and compositions of the present invention provide one or more, and preferably several, unexpected performance properties compared to prior personal care methods, personal care compositions and personal care foams, lotions and gels. These performance advantages include one or more, and preferably at least two of the following:
improved and enhanced sensations to the user, including appearance, feel and/or sound;
improved stability of the emulsion that forms the foam
improved penetration of the active ingredient into the skin, hair and/or mucous membrane of the user
cooling sensation to the skin/mucous membrane that is or at least is perceived to be felt essentially immediately upon application
enhanced and long lasting cooling sensation provided to the user, preferably without the use of sensates.

As used herein, the terms “personal care composition,” personal care formulation” and the like means compositions, formulations and the like that are to be topically applied to a person.

The personal care compositions and formulations of the present invention can be in the form, for example, of lotions, creams, gels and the like.

The personal care compositions and formulations of the present invention include lotions, creams, gels, foams and the like that exhibit post-foaming properties. It will be appreciated, therefore, that while post-foaming formulations are preferred in many applications, lotions, creams, gels and the like that are not post-foaming are also within the broad scope of the present invention, including particularly embodiments which provide cooling sensation to the user. The compositions and formulations of the present invention can be in the form of sprayed or aerosolized powders, mists and the like which generally are not post-foaming compositions but which provide the advantageous cooling sensation of the present invention.

As used herein, the term “post-foaming” refers to foams, gels, creams, lotions and the like which, as a result of the application of heat (which may be in the form of body heat) or other energy (such as kinetic energy associated with rubbing or spreading the formulation on the skin and/or in the hair, for example), produce a foamed product and/or a continuing sensation of a foaming process, such as for example post-application crackling and/or bubbling.

As we be appreciated by those skilled in the art, while the present invention provides several types of foams, including novel foams, and several foaming methods, many ingredients of the components used to form the foam form are used (required and/or optional) are common to all of the formulations. Preferred aspects and examples of these components are described below:

### Active Components

The active component(s) used in the compositions and methods of the present invention can have a wide variety of purposes and functions, and all such components are within the scope of the present invention. In general, such components will provide or at least be expected to provide some benefit to the user, preferably to the skin, hair and/or mucous membrane of the user. Thus, the active components can include cosmetic agents (that is, agents that are cosmetically treating, providing nutrition to and/or conditioning the skin, hair or mucous membrane via topical application), medicinal agents (that is, agents that capable of promoting or assisting in the recovery from injury or illness, preferably to the skin, hair or mucous membrane), and pharmaceutical agents (that is, drugs that are appropriate for delivery by topical application, including preferably drugs that are effective at treating skin, hair and mucous membranes). It will be appreciated by those skilled in the art that active ingredients can have more than one mode of operation and/or effect and therefore may be properly included in two or more categories of product for the purposes of the present invention.

Examples of active components include but are not limited to: coloring, de-coloring and appearance altering agents (such as pigments, depigmentation agents, skin lighteners, reflectants and the like); hair treatment agents (such as shaping agents, detangling agents, wet combing agents, anti-hair loss agents, hair growth promoting agents, hair growth inhibitors, hair colorants, hair conditioners, hair softeners; hair moisturizers, anti-dandruff agents and the like); film forming polymers; humectants; amino acids and their derivatives; antimicrobial agents; allergy inhibitors; anti-acne agents (such as salicylic acid, benzoyl peroxide, sulfur, adapalene and glycolic acid); anti-aging and anti-wrinkling agents (such as dimethylaminoethanol (“DMAE”), retinol, Vitamin C, hydroxy acids, peptides, tea extracts, grape seed extracts and niacinamide), antiseptics; analgesics; antitussives; anti-pruritics; local anesthetics; antihistamines; anti-infective; inflammation inhibitors; deodorants and anti-perspirants (aluminium chlorohydrate and aluminium-zirconium tetrachlorohydrate); medicament agents; skin emollients and skin moisturizers; skin firming agents; anti-fungal agents; shaving preparations, lotions, creams and gels; perfumes; make-up preparations; cosmetics (such as skin cleansers, skin conditioners, make-up and the like); and sun screen and sun blocks (such as benzophenones, bornelone, butyl paba, cinnamidopropyl trimethyl ammonium chloride, disodium distyrylbiphenyl disulfonate, paba, potassium methoxycinnamate, butyl methoxydibenzoylmethane, octyl methoxycinnamate, oxybenzone, octocrylene, octyl salicylate, phenylbenzimidazole sulfonic acid, ethyl hydroxypropyl aminobenzoate, menthyl anthranilate, aminobenzoic acid, cinoxate, diethanolamine methoxycinnamate, glyceryl aminobenzoate, titanium dioxide, zinc oxide, oxybenzone, Padimate 0, and red petrolatum); insect repellants, skin protectants (such as atmeal, betaglucan, feverfew, soy and derivatives thereof, bicarbonate of soda, colloidal oatmeal, surfactant based colloidal oatmeal cleanser, Anagallis Arvensis, Oenothera biennis, Verbena officinalis, and the like), vitamins (such as vitamin B complex; including thiamine, nicotinic acid, biotin, pantothenic acid, choline, riboflavin, vitamin B6, vitamin B12, pyridoxine, inositol, carnitine; vitamins A, C, D, E, K and their derivatives such as vitamin A palmitate and pro-vitamins, e.g. (i.e. panthenol (pro vitamin B5) and panthenol triacetate).

The amount of the active component(s) to be used with the present invention can vary widely depending on the particular application, and all such amounts can be determined by those skilled in the art in view of the teachings contained herein and are within the broad scope of the invention. For example, amount of the active component(s) may vary depending upon the ability of the component to penetrate through, onto, or into the pores of the skin, hair or mucous membrane, in view of the enhancements thereto provided by the present invention. Other factors which may influence the relative amount of the active component include the particular active component involved, the particular benefit desired, the sensitivity of the user to the active component, the health condition, age, and skin, hair, and/or mucous membrane condition of the user, and the like. In sum, the benefit agent is preferably used in a “safe and effective amount,” which is an amount that is high enough to deliver a desired skin, hair or mucous membrane benefit or to modify a certain condition to be treated and which avoids unwanted deleterious serious side effects. In preferred embodiments, the active components together are present in the personal care composition or personal care formulation system in an amount, based upon the total weight of the composition/formulation, of from about 0.01 percent to about 50 percent by weight, or from about 0.01 percent to about 20 percent, or from about 0.01 percent to about 10 percent or from about 0.01 percent to about 5 percent, or from about 0.01 percent to about 1.0 percent by weight.

### Auxiliary Gel, Lotion or Cream Forming Components

The types of auxiliary components that may be used in the formulations of the present invention can vary widely depending on the particular form of the formulation (e.g., gel, cream or lotion) and the particular attributes of the formulation desired, and any and all such components are included within the scope of the present invention.

In connection with gels, for example, the gel forming component in preferred embodiments comprises, preferably in major proportion based on the total weight of the formulation, an aqueous soap. The soap in the aqueous soap component can be, for example, either a soap or a surface active agent, which is also sometimes called a wetting agent, which is generally anionic or nonionic in character.

The soaps (water soluble salts of higher fatty acids) which are preferably amine or basic salts, that are suitable for use in personal care are generally known in the art, and all such soaps are within the scope of the present invention. In general, such a component can preferably be an alkali, ammonium or soluble amine salt of a fatty acid such as stearic acid, palmitic acid, myristic acid and the like. As known in the art, soaps may also be prepared by neutralization or saponification of animal fats, such as tallow or vegetable fats. The selection of a suitable soap component is deemed to be within the scope of those skilled in the art from the teachings herein.

### Non-Ionic Solid Emulsifier

Preferably in the present methods, including each of Methods 1-6, and in the present personal care compositions, including each of Compositions 1-5, the emulsifier comprises or consists essentially of, or consists of, a non-ionic, solid emulsifier. As indicated herein, solid emulsifiers as used herein refers to emulsifiers that are solid at about room temperature or above. The present invention, including each of Methods 1-6 and each of Personal Care Compositions 1-5 includes the use of non-ionic emulsifiers that comprise, consist essentially of or consist of emulsifiers having a normal melting point of about 35° C. or greater and an HLB value of from about 7 to about 18. The present invention, including each of Methods 1-6 and each of Compositions 1-5, includes the use of non-ionic emulsifiers that comprise, consist essentially of or consist of emulsifiers having a normal melting point of from about 35° C. to about 80° C. and an HLB value of from about 7 to about 18.

The present invention, including each of Methods 1-6 and each of Compositions 1-5, includes the use of solid emulsifiers comprising, consisting essentially of or consisting of solid emulsifiers that are long chain, substantially linear emulsifier compounds having an HLB value of from about 7 to about 19, or from about 9 to about 16.

Examples of solid, non-ionic emulsifiers of the present invention, including each of Methods 1-6 and each of Compositions 1-5_that are solid non-ionic emulsifiers that have an HLB value of from about 7 to about 19 and a normal melting point of from about 35° C. to about 80° C. include each of the emulsifiers in the following and mixtures of any two or more of these with each other and/or with other emulsifiers:

| | Exemplary Trade | Melting point | |
|---|---|---|---|
| INCI* Name | Name | (° C.) | HLB |
| CETEARETH-20 | BRIJ CS20 | 40 | 15.7 |
| CETETH-10 | BRIJ C10 | 32-34 | 12.9 |
| CETETH-20 | BRIJ C20 | 32-34 | 15.7 |
| ISOCETETH-20 | BRIJ IC20 | 36 | 15.7 |
| LAURETH-23 | BRIJ L23 | 41-45 | 16.9 |
| STEARETH-10 | BRIJ S10 | 37-39 | 12.4 |
| STEARETH-100 | BRIJ S100 | 51-54 | 18.8 |
| STEARETH-21 | BRIJ S721 | 36 | 15.5 |
| PEG-150 | EUMULGIN ® EO 33 | 52-57 | 17 |
| DISTEARATE | | | |
| PEG-100 STEARATE | ETHOXCARE ® MS- | 54-59 | 17 |
| | 100 | | |
| PEG-150 STEARATE | HALLSTAR ® PEG | 60 | 18 |
| | 6000 MS | | |
| PEG-40 STEARATE | ALKAMULS ® AC- | 44-47 | 16.7 |
| | S40 | | |
| SORBITAN | EMALEX SPE-100S | 54-57 | 8 |
| STEARATE | | | |

*International Nomenclature of Cosmetic Ingredients

The present invention, including each of Methods 1—and each of Personal Care Compositions 1-, also includes the use of solid emulsifiers comprising, consisting essentially of or consisting of solid emulsifiers that are long chain, substantially linear emulsifier compounds having an HLB value of from about 7 to about 19, or from about 9 to about 16 and having the following general molecular structure:

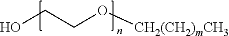

where n is from 10 to 100, or from 10 to about 30, and
m is from 12-22.
   Examples of such an emulsifier include:
   Ceteareth 20 having the formula

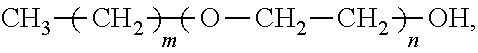

where m=15-17 and n=20, and Steareth 20 (C₂₀H₄₁O₂) and Steareth 21 (poly(oxy-1,2-ethanediyl), .alpha.-octadecyl-.omega.-hydroxy).

### Polymeric Emulsifier

In the present methods, including each of Methods 1-6, and in the present personal care compositions, including each of Compositions 1-6, the polymeric emulsifier polymeric materials, that emulsify and/or stabilize oil-in-water emulsions, preferably at relatively low loadings in the formulation. In the present methods, including each of Methods 1-6, and in the present personal care compositions, including each of Compositions 1-6, the polymeric emulsifier can comprise, consist essentially of or consist of acrylate C10-C30 alkyl-acrylate cross polymer(s). Examples of such polymeric emulsifiers include the emulsifier sold under the trade names Pemulen TR-1 NF and Pemulen TR-2 NF by Lubrizol.

### Other Emulsifiers

In the present methods, including each of Methods 1-6, and in the present personal care compositions, including each of Compositions 1-5, the emulsifier can comprise an emulsifier other than the preferred solid non-ionic emulsifiers. For example, the methods and formulations may utilize liquid non-ionic emulsifiers and/or cationic emulsifiers.

### Thickeners

In the present methods, including each of Methods 1-6, and in the present personal care compositions, including each of Compositions 1-5, the thickener refers to a component or combination of components that tend to thicken the continuous phase (i.e., aqueous phase) and/or the discontinuous phase, that is, to increase the viscosity of those phases and/or the emulsion overall.

In the present methods, including each of Methods 1-6, and in the present personal care compositions, including each of Compositions 1-6, the thickener can comprise, consist essentially of or consist of a solid thickener for the discontinuous phase. While it is contemplated that solid thickeners of the type known to those skilled in the art can be used in the present formulations, in preferred embodiments the solid oil phase thickener comprises one or more of solid fatty alcohol(s), solid cationic emulsifiers, and high melting point waxes.

In preferred embodiments of the present methods, including each of Methods 1-6, and in the present personal care compositions, including each of Compositions 1-6, the solid thickener comprises, consist essentially of or consist of a fatty alcohol. In such embodiments, the fatty alcohol may comprises, for example cetearyl alcohol, cetyl alcohol, behenyl alcohol, isostearyl alcohol and combinations of these. As with the other components useful in the present invention, it will be appreciated that fatty alcohols may, in addition to acting as a thickener for the oil phase, act as a skin moisturizer, emollient or have other desirable functions.

In preferred embodiments of the present methods, including each of Methods 1-6, and in the present personal care compositions, including each of Compositions 1-5, the solid thickener comprises, consist essentially of or consist of a high melting point wax. As used herein, the term “high melting point wax” refers to waxes, and preferably natural waxes, that have a melting point of about 60° C. or higher, or higher than about 70° C. or higher, or higher than about 80° C. or higher. In such embodiments, the high melting point wax may comprise, for example, carnauba wax, and/or candelilla wax and/or bees wax. As with the other components useful in the present invention, it will be appreciated that high melting point wax may, in addition to acting as a thickener for the oil phase, act as an emollient or have other desirable functions.

### Oil Phase Liquids

In preferred embodiments of the present methods, including each of Methods 1-6, and in the present personal care compositions, including each of Compositions 1-5, the formulation includes oil phase liquids other than 1233zd(E) and 1234ze(E). The oil phase liquid can be any liquid component that is acceptable for use in connection with personal care products, and preferably comprises liquid components that have an advantageous functionality in connection with a feature desired by the user, as a moisturizer and/or a protecting agent for the skin or hair of the user. The oil phase liquid may comprise, consist essentially of or consist of argan oil(s), ester oil(s), silicone oils, liquid emulsifiers (non-ionic, anionic, and/or cationic, preferably non-ionic), and combinations of these.

### Polar Carrier

In preferred embodiments of the present methods, including Method 4, and in the present personal care compositions, including Composition 3, the formulation includes a continuous phase that includes a polar carrier having a normal boiling point or an initial boiling point of from about 60° C. to about 200° C. In preferred embodiments of the present methods, including each of Method 4, and in the present personal care compositions, including each of Compositions 3, the formulation includes a continuous phase that includes a polar carrier having a normal boiling point or an initial boiling point of from about 60° C. to about 150° C. In preferred embodiments of the present methods, including each of Method 4, and in the present personal care compositions, including each of Compositions 3, the formulation includes a continuous phase that includes a polar carrier having a normal boiling point or an initial boiling point of from about 60° C. to about 100° C. In preferred embodiments of the present methods, including each of Method 4, and in the present personal care compositions, including each of Compositions 3, the formulation includes a continuous phase that includes a polar carrier having a normal boiling point or an initial boiling point of from about 100° C. to about 200° C. In the broad sense, the polar carrier can be any liquid component that is acceptable for use in connection with personal care products. The polar carrier may comprise, consist essentially of or consist of ethanol, propylene glycol and combinations of these.

### Discontinuous Phase Refractive Index Balancer

In preferred embodiments of the present methods, including each of Method 6, and in the present personal care compositions, including each of Composition 5, the formulation includes a DRIB. In preferred embodiments of the present methods, including each of Method 6, and in the present personal care compositions, including each of Compositions 5, the formulation includes a DRIB that comprises, consists essentially of or consist of a siloxane and/or a benzoate suitable for use in personal care products. In preferred embodiments of the present methods, including each of Method 6, and in the present personal care compositions, including each of Composition 5, the formulation includes a DRIB that comprises, consists essentially of or consist of a cycloalkyl siloxane, preferably a cyclopentasiloxane and/or a cyclohexasiloxane. Examples of such a siloxane include the material sold under the trade designation XIAMETER PMX-0345-345 by Dow. In preferred embodiments of the present methods, including each of Method 6, and in the present personal care compositions, including each of Composition 5, the formulation includes a DRIB that comprises, consists essentially of or consist of a C12-C15 alkyl benzoate. Examples of such a benzoate include the material sold under the trade designation TEGOSOFT TN by Evonik.

### Continuous Phase Refractive Index Balancer

In preferred embodiments of the present methods, including each of Method 6, and in the present personal care compositions, including each of Composition 5, the formulation includes a CRIB. In preferred embodiments of the present methods, including each of Method 6, and in the present personal care compositions, including each Compositions 5, the formulation includes a CRIB that comprises, consists essentially of or consist of one or more polyols suitable for use in personal care products. In preferred embodiments of the present methods, including each of Method 6, and in the present personal care compositions, including each of Composition 5, the formulation includes a CDRIB that comprises, consists essentially of or consist of glycerine, polyethylene glycol and combinations of these. Examples of such polyethylene glycol include ERCAPEG 200 made by Erca Wilmar.

### Other Auxiliary Components

Other auxiliary ingredients that may be included in the present gels, creams and lotions for use in post-foaming applications include polymers for gel thickening and lubrication, cosmetic esters for emolliency, humectants for moisturization, fragrances, dyes and preservatives.

The amount of the auxiliary component(s) to be used with the present invention can vary widely depending on the particular application, and all such amounts can be determined by those skilled in the art in view of the teachings contained herein and are within the broad scope of the invention. In preferred embodiments, the auxiliary component(s) together are present in the personal care composition or personal care formulation system in an amount, based upon the total weight of the composition/formulation, of from about 1 percent to about 60 percent based on the total weight of the components in formulation.

### Ice Cream Formulations

The present invention includes each of Methods 1 and 2, and in the present personal care compositions, including each of Compositions 1, wherein the solid thickener comprises, consists essentially of, or consists of solid fatty alcohol and/or solid cationic emulsifier and/or a high melting point wax, and combinations of these. For the purposes of convenience, a method according to the present paragraph is referred to herein as Method 2A. For the purposes of convenience, a composition according to the present paragraph is referred to herein as Composition 1A.

The present invention includes each of Methods 1 and 2, and in the present personal care compositions, including each of Compositions 1, wherein the solid thickener comprises, consists essentially of, or consists of solid fatty alcohol. For the purposes of convenience, a method according to the present paragraph is referred to herein as Method 2B. For the purposes of convenience, a composition according to the present paragraph is referred to herein as Composition 1B.

The present invention includes each of Methods 1 and 2, and in the present personal care com-positions, including each of Compositions 2, wherein the solid thickener comprises, consists essentially of, or consists of a high melting point wax. For the purposes of convenience, a method according to the present paragraph is referred to herein as Method 2C. For the purposes of convenience, a composition according to the present paragraph is referred to herein as Composition 1C.

The present invention includes each of Methods 1 and 2, and in the present personal care com-positions, including each of Compositions 2, wherein the solid thickener comprises, consists essentially of, or consists of a solid cationic emulsifier. For the purposes of convenience, a method according to the present paragraph is referred to herein as Method 2D. For the purposes of convenience, a composition according to the present paragraph is referred to herein as Composition 1D.

The present invention includes each of Methods 1 and 2, and present personal care com-positions, including each Compositions 2, wherein an aqueous phase thickener is present and wherein the weight ratio of the aqueous phase thickener to the oil phase thickener is less than 0.015. For the purposes of convenience, a method according to the present paragraph is referred to herein as Method 2E. For the purposes of convenience, a composition according to the present paragraph is referred to herein as Composition 1E.

The present invention includes each of Methods 1 and 2, and present personal care com-positions, including each Compositions 2, wherein said 1233zd(E) and said 1234ze (E) together comprise from about 40% by weight to about 60% by weight of said personal care composition For the purposes of convenience, a method according to the present paragraph is referred to herein as Method 2F. For the purposes of convenience, a composition according to the present paragraph is referred to herein as Composition 1F.

The present invention includes each of Methods 1 and 2, and present personal care com-positions, including each of Compositions 2, wherein said 1233zd(E) and said 1234ze (E) are present in a trans1233zd:trans1234ze weight ratio of from about 1:2 to about 1:6, or from 1:2 to about 1:4, or about 1:4 For the purposes of convenience, a method according to the present paragraph is referred to herein as Method 2G. For the purposes of convenience, a composition according to the present paragraph is referred to herein as Composition 1G.

The present invention includes each of Methods 1 and 2, and present personal care com-positions, including each of Compositions 2, wherein the net HLB of the formulation is from about 7 to about 16, or from about 8 to about 16. For the purposes of convenience, a method according to the present paragraph is referred to herein as Method 2H. For the purposes of convenience, a composition according to the present paragraph is referred to herein as Composition 1H.

The present invention includes each of Methods 1 and 2, and present personal care com-positions, including each of Compositions 2, wherein at least 50% by weight of the emulsifier comprises one or more solid non-ionic emulsifier(s) with an HLB of at least about 10, or from about 10 to about 18, or from about 7 to about 16, or from about 8 to about 16. For the purposes of convenience, a method according to the present paragraph is referred to herein as Method 21. For the purposes of convenience, a composition according to the present paragraph is referred to herein as Composition 1I.

The present invention includes each of Methods 1 and 2, and present personal care com-positions, including each of Compositions 2, wherein the weight ratio of said oil phase thickeners to said total oil phase liquids is from about 2 to about 3. For the purposes of convenience, a method according to the present paragraph is referred to herein as Method 2J. For the purposes of convenience, a composition according to the present paragraph is referred to herein as Composition 1J.

### Sherbet Formulations

The present invention includes each of Methods 1 and 3, and in the present personal care compositions, including each of Compositions 2, wherein the solid thickener comprises, consists essentially of, or consists of solid fatty alcohol and/or solid cationic emulsifier and/or a high melting point wax, and combinations of these. For the purposes of convenience, a method according to the present paragraph is referred to herein as Method 3A. For the purposes of convenience, a composition according to the present paragraph is referred to herein as Composition 2A.

The present invention includes each of Methods 1 and 3, and in the present personal care compositions, including each of Compositions 2, wherein the solid thickener comprises, consists essentially of, or consists of solid fatty alcohol. For the purposes of convenience, a method according to the present paragraph is referred to herein as Method 3B. For the purposes of convenience, a composition according to the present paragraph is referred to herein as Composition 2B.

The present invention includes each of Methods 1 and 3, and in the present personal care com-positions, including each of Compositions 3, wherein the solid thickener comprises, consists essentially of, or consists of a high melting point wax. For the purposes of convenience, a method according to the present paragraph is referred to herein as Method 3C. For the purposes of convenience, a composition according to the present paragraph is referred to herein as Composition 2C.

The present invention includes each of Methods 1 and 3, and in the present personal care com-positions, including each of Compositions 2, wherein the solid thickener comprises, consists essentially of, or consists of a solid cationic emulsifier. For the purposes of convenience, a method according to the present paragraph is referred to herein as Method 3D. For the purposes of convenience, a composition according to the present paragraph is referred to herein as Composition 2D.

The present invention includes each of Methods 1 and 3, and present personal care com-positions, including each Compositions 2, wherein an aqueous phase thickener is present. For the purposes of convenience, a method according to the present paragraph is referred to herein as Method 3E. For the purposes of convenience, a composition according to the present paragraph is referred to herein as Composition 2E.

The present invention includes each of Methods 1 and 3, and present personal care com-positions, including each Compositions 2, wherein said 1233zd(E) and said 1234ze (E) together comprise from about 40% by weight to about 60% by weight of said personal care composition For the purposes of convenience, a method according to the present paragraph is referred to herein as Method 3F. For the purposes of convenience, a composition according to the present paragraph is referred to herein as Composition 2F.

The present invention includes each of Methods 1 and 3, and present personal care com-positions, including each of Compositions 2, wherein said 1233zd(E) and said 1234ze (E) are present in a trans1233zd:trans1234ze weight ratio of from about 6:1 to 1:1, or from about 4:1 to 1:1, or about 4:1 to about 2:1 For the purposes of convenience, a method according to the present paragraph is referred to herein as Method 3G. For the purposes of convenience, a composition according to the present paragraph is referred to herein as Composition 2G.

The present invention includes each of Methods 1 and 3, and present personal care com-positions, including each of Compositions 2, wherein the net HLB of the formulation is from about 7 to about 16, or from about 8 to about 16. For the purposes of convenience, a method according to the present paragraph is referred to herein as Method 3H. For the purposes of convenience, a composition according to the present paragraph is referred to herein as Composition 2H.

The present invention includes each of Methods 1 and 3, and present personal care com-positions, including each of Compositions 2, wherein at least 50% by weight of the emulsifier comprises one or more non-ionic emulsifier(s) with an HLB of at least about 10, or from about 10 to about 18, or from about 7 to about 16, or from about 8 to about 16. For the purposes of convenience, a method according to the present paragraph is referred to herein as Method 31. For the purposes of convenience, a composition according to the present paragraph is referred to herein as Composition 21.

The present invention includes each of Methods 1 and 3, and present personal care com-positions, including each of Compositions 2, wherein at least 50% by weight of the emulsifier comprises one or more solid non-ionic emulsifier(s) with an HLB of at least about 10, or from about 10 to about 18, or from about 7 to about 16, or from about 8 to about 16. For the purposes of convenience, a method according to the present paragraph is referred to herein as Method 3J. For the purposes of convenience, a composition according to the present paragraph is referred to herein as Composition 2J.

### Ultra-Crackling Foam

The present invention includes each of Methods 1 and 4, and Composition 3, wherein the polar carrier comprises, consists essentially of, or consists of ethanol. For the purposes of convenience, a method according to the present paragraph is referred to herein as Method 4A. For the purposes of convenience, a composition according to the present paragraph is referred to herein as Composition 3A.

The present invention includes each of Methods 1 and 4, and Composition 3, wherein the polar carrier comprises, consists essentially of, or consists of propylene glycol. For the purposes of convenience, a method according to the present paragraph is referred to herein as Method 4B. For the purposes of convenience, a composition according to the present paragraph is referred to herein as Composition 3B.

The present invention includes each of Methods 1 and 4, and Compositions 3, wherein the personal care composition comprises a solid oil phase thickener that comprises, consists essentially of, or consists of a fatty alcohol and/or a high melting point wax. For the purposes of convenience, a method according to the present paragraph is referred to herein as Method 4C. For the purposes of convenience, a composition according to the present paragraph is referred to herein as Composition 3C.

The present invention includes each of Methods 1 and 4, and Composition 2, wherein the personal care composition comprises an aqueous phase thickener. For the purposes of convenience, a method according to the present paragraph is referred to herein as Method 4D. For the purposes of convenience, a composition according to the present paragraph is referred to herein as Composition 3D.

The present invention includes each of Methods 1 and 3, and Composition 3, wherein said 1233zd(E) and said 1234ze (E) together comprise from about 40% by weight to about 80% by weight of said personal care composition. For the purposes of convenience, a method according to the present paragraph is referred to herein as Method 4E. For the purposes of convenience, a composition according to the present paragraph is referred to herein as Composition 3E.

The present invention includes each of Methods 1 and 4, and Composition 3, wherein said 1233zd(E) and said 1234ze (E) are present in a trans1233zd:trans1234ze weight ratio of from about 1:12 to 1:4, or from about 1:10 to 1:4, or about 1:8 to about 1:4 For the purposes of convenience, a method according to the present paragraph is referred to herein as Method 4F. For the purposes of convenience, a composition according to the present paragraph is referred to herein as Composition 3F.

The present invention includes each of Methods 1 and 4, and Composition 3, wherein the net HLB of the formulation is from about 7 to about 16, or from about 8 to about 16. For the purposes of convenience, a method according to the present paragraph is referred to herein as Method 4G. For the purposes of convenience, a composition according to the present paragraph is referred to herein as Composition 3G.

The present invention includes each of Methods 1 and 4, and Composition 3, wherein at least 50% by weight of the emulsifier comprises one or more liquid non-ionic emulsifier(s) with an HLB of at least about 10, or from about 10 to about 18, or from about 7 to about 16, or from about 8 to about 16. For the purposes of convenience, a method according to the present paragraph is referred to herein as Method 4H. For the purposes of convenience, a composition according to the present paragraph is referred to herein as Composition 3H.

The present invention includes each of Methods 1 and 4, and Composition 3, wherein at least 50% by weight of the emulsifier comprises one or more liquid non-ionic emulsifier(s) with an HLB of at least about 10, or from about 10 to about 18, or from about 7 to about 16, or from about 8 to about 16. For the purposes of convenience, a method according to the present paragraph is referred to herein as Method 41. For the purposes of convenience, a composition according to the present paragraph is referred to herein as Composition 31.

### Bubbling Foam

The present invention includes each of Methods 1 and 5, and Composition 4, wherein the polymeric emulsifier comprises, consists essentially of, or consists of acrylate-based copolymer. For the purposes of convenience, a method according to the present paragraph is referred to herein as Method 5A. For the purposes of convenience, a composition according to the present paragraph is referred to herein as Composition 4A.

The present invention includes each of Methods 1 and 5, and Composition 4, wherein the polymeric emulsifier comprises, consists essentially of, or consists of C10-C30 alkyl-acrylate cross polymer(s). For the purposes of convenience, a method according to the present paragraph is referred to herein as Method 5B. For the purposes of convenience, a composition according to the present paragraph is referred to herein as Composition 4B.

The present invention includes each of Methods 1 and 5, and Composition 4, wherein the weight ratio of all the components in the oil phase to said polymeric emulsifier is from about 530 to about 675. For the purposes of convenience, a method according to the present paragraph is referred to herein as Method 5C. For the purposes of convenience, a composition according to the present paragraph is referred to herein as Composition 4C.

The present invention includes each of Methods 1 and 5, and Composition 4, wherein said 1233zd(E) and said 1234ze (E) together comprise from about 40% by weight to about 80% by weight of said personal care composition. For the purposes of convenience, a method according to the present paragraph is referred to herein as Method 5D. For the purposes of convenience, a composition according to the present paragraph is referred to herein as Composition 4D.

The present invention includes each of Methods 1 and 5, and Composition 4, wherein said 1233zd(E) and said 1234ze (E) are present in a trans1233zd:trans1234ze weight ratio of from about 4:1 to 1:1. For the purposes of convenience, a method according to the present paragraph is referred to herein as Method 5E. For the purposes of convenience, a composition according to the present paragraph is referred to herein as Composition 4E.

The present invention includes each of Methods 1 and 5, and Composition 4, wherein the net HLB of the formulation is from about 7 to about 16, or from about 8 to about 16. For the purposes of convenience, a method according to the present paragraph is referred to herein as Method 5F. For the purposes of convenience, a composition according to the present paragraph is referred to herein as Composition 4E.

The present invention includes each of Methods 1 and 5, and Composition 4, wherein at least 50% by weight of the emulsifier comprises polymeric emulsifier. For the purposes of convenience, a method according to the present paragraph is referred to herein as Method 5G. For the purposes of convenience, a composition according to the present paragraph is referred to herein as Composition 4G.

### Clear Foam, Gel and Lotion

The present invention includes each of Methods 1 and 6, and Composition 5, wherein the said discontinuous phase comprises a discontinuous phase refractive index balancer (DRIB) in an amount effective to produce a weight ratio of said 1233zd(E):DRIB of from about 4:1 to about 25:1. For the purposes of convenience, a method according to the present paragraph is referred to herein as Method 6A. For the purposes of convenience, a composition according to the present paragraph is referred to herein as Composition 5A.

The present invention includes each of Methods 1 and 6, and Composition 5, wherein the said discontinuous phase comprises a discontinuous phase refractive index balancer (DRIB) in an amount effective to produce a weight ratio of said 1233zd(E):DRIB of from about 5:1 to about 20:1. For the purposes of convenience, a method according to the present paragraph is referred to herein as Method 6B. For the purposes of convenience, a composition according to the present paragraph is referred to herein as Composition 5B.

The present invention includes each of Methods 1 and 6, and Composition 5, wherein the DRBI comprises, consists essentially of, or consists of a siloxane and/or a benzoate. For the purposes of convenience, a method according to the present paragraph is referred to herein as Method 6C. For the purposes of convenience, a composition according to the present paragraph is referred to herein as Composition 5C.

The present invention includes each of Methods 1 and 6, and Composition 5, wherein the said discontinuous phase comprises a discontinuous phase refractive index balancer (DRIB and the continuous phase includes a continuous phase refractive index balancer (CRIB). For the purposes of convenience, a method according to the present paragraph is referred to herein as Method 6D. For the purposes of convenience, a composition according to the present paragraph is referred to herein as Composition 5D.

The present invention includes each of Methods 1 and 6, and Composition 5, wherein the said foam, gel or lotion has a refractive index of from about 1.33. For the purposes of convenience, a method according to the present paragraph is referred to herein as Method 6E. For the purposes of convenience, a composition according to the present paragraph is referred to herein as Composition 5E.

The present invention includes each of Methods 1 and 6, and Composition 5, wherein wherein said DRIB is present in an amount effective to produce a refractive index of the discontinuous phase equal to the refractive index of the continuous phase+/−0.005, wherein the weight ratio of said 1233zd(E):DRIB is from about 3:1 to about 15:1. For the purposes of convenience, a method according to the present paragraph is referred to herein as Method 6F. For the purposes of convenience, a composition according to the present paragraph is referred to herein as Composition 5F.

The present invention includes each of Methods 1 and 6, and Composition 5, wherein wherein said DRIB is present in an amount effective to produce a refractive index of the discontinuous phase equal to the refractive index of the continuous phase+/−0.005. For the purposes of convenience, a method according to the present paragraph is referred to herein as Method 6G. For the purposes of convenience, a composition according to the present paragraph is referred to herein as Composition 5G.

The present invention includes each of Methods 1 and 6, and Composition 5, wherein wherein wherein said DRIB has a refractive index of from about 1.375 to about 1.7. For the purposes of convenience, a method according to the present paragraph is referred to herein as Method 6H. For the purposes of convenience, a composition according to the present paragraph is referred to herein as Composition 5H.

The present invention includes each of Methods 1 and 6, and Composition 5, wherein said aqueous phase further comprises one or more continuous phase refractive index balancers (CRIB) in an amount effective to produce a refractive index of the continuous phase of from about 1.34 to about 1.37. For the purposes of convenience, a method according to the present paragraph is referred to herein as Method 61. For the purposes of convenience, a composition according to the present paragraph is referred to herein as Composition 51.

The present invention includes each of Methods 1 and 6, and Composition 5, wherein said 1233zd(E) is present in the oil-in-water emulsion in an amount of 20% by weight or less based on all the components in the oil-in-water emulsion. For the purposes of convenience, a method according to the present paragraph is referred to herein as Method 6J. For the purposes of convenience, a composition according to the present paragraph is referred to herein as Composition 5J.

The present invention includes each of Methods 1 and 6, and Composition 5, wherein said emulsifier comprises a copolymer comprising an acrylate moiety and/or a taurate moiety. For the purposes of convenience, a method according to the present paragraph is referred to herein as Method 6K. For the purposes of convenience, a composition according to the present paragraph is referred to herein as Composition 5K.

### Personal Care Uses

The present personal care compositions can be in the form of and/or used in a wide variety of personal care products/applications, including: shampoos (including baby shampoos, conditioning shampoos, moisturizing shampoos, etc.); skin and body cleansers (including shower gels, bath foams, facial cleansers, intimate cleansers, moisturizing body washes, antibacterial body washes; bath gels; hand soaps; facial scrubs; foot scrubs, etc); hair care compositions (including hair dyes, hair conditioners, hair creams and hair styling formulations); skin care compositions (including skin lotions, balms and creams, skin whiteners, self tanning lotions, sunscreen lotions, carrier lotions, moisturizers, vitamin C creams, antibacterial lotions, facial masks, body masks, make up foundations, sun care formulations, antiperspirants, etc).

### EXAMPLES

In the examples which follow, the following components are used and, unless otherwise specifically indicated in the example, have at least the function indicated below:

| | Identifier/Trade | |
|---|---|---|
| Ingredient | name/Abreviation | Function |
| water | | Solvent |
| Glycerin (water phase) | | humectant |
| Dimethicon (oil phase - liquid) | | conditioning agent |
| Cyclopentasiloxane/Cyclopentasiloxane | Xiameter PMX-0345 | spreading agent |
| blend (oil phase - liquid) | | |
| Citric Acid (water phase) | | pH adjuster |
| Argan Oil (oil phase) | | Skin softener |
| Chlorphenesin /phenoxyethanol (oil phase) | | Preservative |
| Behentrimonium Chloride (oil phase) | BTAC DPG 70 | Solid Cationic |
| | | emulsifier |
| Brij CS20 − HLB = 15.7 (oil phase) | | Solid Non-ionic |
| | | emulsifier |
| Glyceryl Stearate − HLB = 3.8 (oil phase) | | Non-ionic Emulsifier |
| | | (solid) |
| Glycol Distearate − HLB = 1 (oil phase) | | Non-ionic Emulsifier |
| | | (solid) |
| Cetearyl Alcohol (oil phase) - Fatty alcohol | Lanette O | Thickener (also skin |
| | | softener) |
| Cetyl Alcohol (oil phase) Fatty alcohol | Lanette 16 | Thickener (also skin |
| | | softener) |
| Fragrance | 242091 GB BLDELI | Fragrance |
| Red 4/Yellow 6 | Colorant | Red 4/Yellow 6 |

The examples which follow also provide several weight ratios values. For the purposes of convenience, the following abbreviations are used.

| Weight Ratio | Abbreviation |
|---|---|
| Cationic:NonIonic Emulsifier | Cat:NonI |
| Ratio of solid non-ionic emulsifier to total | SNIE:TOPT |
| oil phase thickener | |
| Ratio of solid non-ionic emulsifier to Total | SNIE:TOP |
| Oil phase | |
| Ratio of sold non-ionic emulsifier to total | SNIE:TOPXZDZE |
| oil phase excluding 1233zd and 1234ze | |
| Ratio of all solids to total oil phase | AS:TOPXZDZE |
| (excluding 1233zd and 1234ze) | |
| Total oil phase liquids (excluding 1233zd | TOPLXZDZE |
| and 1234ze) | |
| Ratio of oil phase thickener to total oil | OPT:TOPLXZDZE |
| phase liquids (excluding 1233zd and 1234zd) | |
| Ratio of water based thickener to oil based | WBT:OBT |
| thickener | |
| Ratio of total oil phase liquids (excluding | TOPLXZDZE:AOPS |
| 1233zd and 1234zd) to all oil phase solids | |
| 1233zd(E)/1234ze(E) wt ratio | ZD:ZE |

### Example 1—Hydrating Body Lotion to Form Ice Cream Foam

Three hydrating body formulations in the form of oil-in-water emulsions were prepared using the components as indicated in Table 1A below. The first formulation 1A is comparative, as explained below. The other two formulations were stable and formed ice cream foam.

TABLE 1

| | Wt % Component | | |
|---|---|---|---|
| | Comparative | | |
| Ingredient | 1A | 1B | 1C |
| water | 41.67 | 40.67 | 40.165 |
| ACTIVE COMPONENTS | | | |
| Glycerin | 1.5 | 1.5 | 1.5 |
| Dimethicon | 0.5 | 0.5 | 0.5 |
| Xiameter PMX-0345 | 0.5 | 0.5 | 0.5 |
| Citric Acid | 0.025 | 0.025 | 0.025 |
| Argan Oil | 1 | 1 | 1 |
| Chlorphenesin/phenoxyethanol | 0.35 | 0.35 | 0.35 |
| EMULISIFIERS | | | |
| BTAC DPG 70 (oil phase) | 1 | 1 | 1 |
| Brij CS20 − HLB = 15.7 | 0.4 | 0.4 | 0.4 |
| Glyceryl Stearate | 0.25 | 0.25 | 0.25 |
| Glycol Distearate | 0.25 | 0.25 | 0.25 |
| (Lanette O) (oil phase) | 1.5 | 2.5 | 2.5 |
| Lanette 16 (oil phase) | 1 | 1 | 1 |
| Fragrance | 0.05 | 0.05 | 0.05 |
| Red 4/Yellow 6 | 0.01 | 0.01 | 0.01 |
| 1233zd | 10 | 10 | 10 |
| 1234ze | 40 | 40 | 40 |
| Total | 100 | 100 | 100 |
| Emulsion Stability | Stable | Stable | Stable |
| Foam Form | Not ice | Ice cream | Ice cream |
| | cream | foam | foam |
| Total Oil phase, wt % of | 56.4 | 57.4 | 57.9 |
| Formulation | | | |
| Total Emulsifier, wt % of | 1.9 | 1.9 | 1.9 |
| Formulation | | | |
| Total non-ionic emulsifiers, | 0.9 | 0.9 | 0.9 |
| wt % of Formulation | | | |
| HLB of non-ionic emulsifiers | 9.56 | 9.56 | 9.56 |
| in Formulation | | | |
| Wt % of Non-ionic emulsifier, | 47.4% | 47.4% | 47.4% |
| based on total emulsifier | | | |
| Cat:NonI | 1.11 | 1.11 | 1.11 |
| Total oil phase thickener | 2.5 | 3.5 | 4 |
| (Fatty Acid Alcohol), wt % | | | |
| Formulation | | | |
| Total oil phase (excluding | 6.48 | 6.4 | 7.9 |
| 1233zd and 1234ze) | | | |
| SNIE:TOPT | 0.360 | 0.257 | 0.225 |
| SNIE:TOP | 0.029 | 0.028 | 0.027 |
| SNIE:TOPXZDZE | 0.14 | 0.12 | 0.11 |
| Total solids in oil phase | 4.48 | 5.4 | 5.9 |
| AS:TOPXZDZE | 0.69 | 0.73 | 0.75 |
| Total oil phase liquids | 2.5 | 3.5 | 4 |
| (excluding 1233zd and 1234ze) | | | |
| OPT:TOPLXZDZE | 1.25 | 1.75 | 2 |
| WBT:OBT | 0 | 0 | 0 |
| TOPLXZDZE:AOPS | 0.45 | 0.37 | 0.34 |
| zd/ze | 1:4 | 1:4 | 1:4 |

The comparative formulation 1A produced a stable oil-in-water emulsion but did not produce ice cream foam—a whipped cream type of foam was formed. In this regard it is noted that Formulation Example 1A used a relative low amount of fatty acid in the formulation (2.5%) and a relatively low amount of total oil phase (exclusive of 1233zd and 1234 ze) (6.4%). This produced relatively high ratios of total solid, non-ionic emulsifier to total fatty acid alcohol (0.14) and a high ratio of oil phase liquids (excluding 1233zd and 1234ze) to oil phase solids (0.45). In contrast, Formulations 1B and 1C were not only stable, but produced ice cream foam. In each of these formulations, a relatively higher amount of fatty acid alcohol was used in the formulation (3.5% and 4%) and a hence a relatively higher amount of total oil phase (exclusive of 1233zd and 1234 ze) (7.4% and 7.9%)). This produced relatively lower ratios of total solid, non-ionic emulsifier to total fatty acid alcohol (0.12 and 0.11) and lower ratio of oil phase liquids (excluding 1233zd and 1234ze) to oil phase solids (0.37 and 0.34, respectively. It is noted that in each of Formulation 1B and 1C the formulation had the following characteristics:

Total of 1233zd plus 1234ze>40 wt % of the formulation
Ratio of 1233zd:1234ze=from 1:8 to 1:2
Net HLB of Solid Non-Ionic Emulsifier within Range—7-18
Total Oil Content (exclusive of 1233zd and 1234zd)—7 wt % or higher
Weight Ratio of Solid Non-Ionic Emulsifier:Total Oil Phase—(ex 1233zd+1234ze)-<0.14.
Weight Ratio of Oil Phase Thickener to all solids in oil phase (ex 1233zd+1234ze)->0.57.
Weight Ratio of Oil Phase Thickener to all liquids in oil phase (ex 1233zd+1234ze)->1.25 and about 3 or less
Weight Ratio of Oil Phase liquids (excluding 1233zzd and 1234ze) to all solids in oil phase<0.45.

There is no water-based thickener present in the formulation.

### Examples 1PA-1PC—Hydrating Body Lotion to Form Ice Cream Foam

Three hydrating body formulation similar to the formulation used in Examples 1B and 1C are formed, water based thickener are used as indicated in Table 1P1 below. Ice cream foams are formed in each case.

TABLE 1P1

| | | Wt % Component | | |
|---|---|---|---|---|
| Ingredient | Function | 1PA | 1PB | 1PC |
| water | Solvent | 40.12 | 39.12 | 38.12 |
| ACTIVE | | | | |
| COMPONENTS | | | | |
| Glycerin (water phase) | Active ingredient - | 1.5 | 1.5 | 1.5 |
| | humectant | | | |
| Dimethieon (oil phase - | Conditioning agent | 0.5 | 0.5 | 0.5 |
| liquid) | | | | |
| Xiameter PMX-0345 | Spreading agent | 0.5 | 0.5 | 0.5 |
| Citric Acid (water phase) | pH adjuster | 0.025 | 0.025 | 0.025 |
| Argan Oil (oil phase) | Skin softener | 1 | 1 | 1 |
| Chlorphenesin/phenoxyethanol | Preservative | 0.35 | 0.35 | 0.35 |
| (oil phase) | | | | |
| EMULISIFIERS | | | | |
| BTAC DPG 70) | Solid Cationic | 1 | 1 | 1 |
| | emulsifier | | | |
| Brij CS20 − HLB = 15.7 | Solid Non-ionic | 0.4 | 0.4 | 0.4 |
| (oil phase) | emulsifier | | | |
| Glyceryl Stearate (HLB = 3.8 | Non-ionic Emulsifier | 0.25 | 0.25 | 0.25 |
| (oil phase) | (solid) | | | |
| Glycol Distearate HLB = 1 | Non-ionic Emulsifier | 0.25 | 0.25 | 0.25 |
| (oil phase) | (solid) | | | |
| Oil phase thickener | (also skin softener) | 4 | 5 | 65 |
| Water phase thickener | | 0.05 | 0.05 | 0.05 |
| Fragrance (242091 GB BLDELI) | Fragrance | 0.05 | 0.05 | 0.05 |
| Colorant (Red 4/Yellow 6) | Red 4/Yellow 6 | 0.01 | 0.01 | 0.01 |
| 1233zd | | 10 | 10 | 10 |
| 1234ze | | 40 | 40 | 40 |
| Total | | 100 | 100 | 100 |
| Emulsion Stability | | Stable | Stable | Stable |
| Foam Form | | Ice cream | Ice cream | Ice cream |
| | | foam | foam | foam |
| Total Oil phase, wt % of | | 57.9 | 58.95 | 59.95 |
| Formulation | | | | |
| Total Emulsifier, wt % of | | 1.9 | 1.9 | 1.9 |
| Formulation | | | | |
| Total non-ionic emulsifiers, | | 0.9 | 0.9 | 0.9 |
| wt % of Formulation | | | | |
| HLB of non-ionic emulsifiers | | 9.56 | 9.56 | 9.56 |
| in Formulation | | | | |
| Wt % of Non-ionic emulsifier, | | 47.4% | 47.4% | 47.4% |
| based on total emulsifier | | | | |
| Ratio Cationic:NonIonic | | 1.11 | 1.11 | 1.11 |
| Emulsifier | | | | |
| Total oil phase thickener | | 4 | 5 | 6 |
| (Fatty Acid Alcohol), wt % | | | | |
| Formulation | | | | |
| Total oil phase (excluding | | 7.9 | 8.9 | 9.9 |
| 1233zd and 1234ze) | | | | |
| Ratio of solid non-ionic | | 0.225 | 0.225 | 0.225 |
| emulsifier to Total oil phase | | | | |
| thickener (Fatty Acid Alcohol) | | | | |
| Ratio of solid non-ionic | | 0.016 | 0.015 | 0.015 |
| emulsifier to Total Oil phase | | | | |
| Ratio of sold non-ionic | | 0.11 | 0.10 | 0.09 |
| emulsifier to total oil phase | | | | |
| excluding 1233zd and 1234ze | | | | |
| Total solids in oil phase | | 5.9 | 6.9 | 7.9 |
| Ratio of all solids to total | | 0.75 | 0.78 | 0.80 |
| oil phase (excluding 1233zd | | | | |
| and 1234ze) | | | | |
| Total oil phase liquids | | 2 | 2 | 2 |
| (excluding 1233zd and 1234ze) | | | | |
| Ratio of oil phase thickener | | 2 | 2.5 | 3 |
| oil phase liquids (excluding | | | | |
| 1233zd and 1234zd) | | | | |
| Ratio of water based thickener | | 0.013 | 0.010 | 0.008 |
| to oil based thickener | | | | |
| Ratio of oil phase liquids | | 0.34 | 0.29 | 0.25 |
| (excluding 1233zd and 1234zd) | | | | |
| to all oil phase solids | | | | |
| 1233zd(E)/1234ze(E) wt ratio | | 1:4 | 1:4 | 1:4 |

Each of the formulation 1PA, 1PB and 1PC produce a stable oil-in-water emulsion and produce ice cream foam. It is noted that each of Formulation had the following characteristics:

Total of 1233zd plus 1234ze>40 wt % of the formulation
Ratio of 1233zd:1234ze=from 1:8 to 1:2
Net HLB of Solid Non-Ionic Emulsifier within Range—7-18
Total Oil Content (exclusive of 1233zd and 1234zd)—7 wt % or higher
Weight Ratio of Solid Non-Ionic Emulsifier:Total Oil Phase—(ex 1233zd+1234ze)-<0.14.
Weight Ratio of Oil Phase Thickener to all solids in oil phase (ex 1233zd+1234ze)->0.57.
Weight Ratio of Oil Phase Thickener to all liquids in oil phase (ex 1233zd+1234ze)->1.25 and about 3 or less.
Weight Ratio of Oil Phase liquids (excluding 1233zzd and 1234ze) to all solids in oil phase<0.45.
Weight Ratio of Water Phase Thickener to oil phase thickener<0.2.

### Examples 1PD-1PF—Hydrating Body Lotion to Form Ice Cream Foam

Three hydrating body formulation similar to the formulation used in Examples 1B and 1C are formed, except with increased amounts of 1233zd(E) and 1234ze(E) while maintaining a 1:4 ratio as indicated in Table 1P2 below. Ice cream foams are formed in each case.

TABLE 1P2

| | | Wt % Component | | |
|---|---|---|---|---|
| Ingredient | Function | 1PD | 1PE | 1PF |
| water | Solvent | 35.17 | 30.17 | 25.17 |
| ACTIVE COMPONENTS | | | | |
| Glycerin (water phase) | Active ingredient - | 1.5 | 1.5 | 1.5 |
| | humectant | | | |
| Dimethicon (oil phase - | Conditioning agent | 0.5 | 0.5 | 0.5 |
| liquid) | | | | |
| Cyclopentasiloxane/ | Spreading agent | 0.5 | 0.5 | 0.5 |
| Cyclopentasiloxane | | | | |
| (oil phase - liquid) | | | | |
| (Xiameter PMX-0345) | | | | |
| Citric Acid | pH adjuster | 0.025 | 0.025 | 0.025 |
| Argan Oil (oil phase) | Skin softener | 1 | 1 | 1 |
| Chlorphenesin/phenoxyethanol | Preservative | 0.35 | 0.35 | 0.35 |
| (oil phase) | | | | |
| EMULLSIFIERS | | | | |
| Behentrimonium Chloride (BTAC | Solid Cationic | 1 | 1 | 1 |
| DPG 70) (oil phase) | emulsifier | | | |
| Brij CS20 − HLB = 15.7 | Solid Non-ionic | 0.4 | 0.4 | 0.4 |
| (oil phase) | emulsifier | | | |
| Glyceryl Stearate (HLB = 3.8 | Non-ionic Emulsifier | 0.25 | 0.25 | 0.25 |
| (oil phase) | (solid) | | | |
| Glycol Distearate HLB = 1 | Non-ionic Emulsifier | 0.25 | 0.25 | 0.25 |
| (oil phase) | (solid) | | | |
| Oil phase thickener | (also skin softener) | 4 | 4 | 4 |
| Water phase thickener | | 0 | 0 | 0 |
| Fragrance (242091 GB BLDELI) | Fragrance | 0.05 | 0.05 | 0.05 |
| Colorant (Red 4/Yellow 6) | Red 4/Yellow 6 | 0.01 | 0.01 | 0.01 |
| 1233zd | | 10 | 10 | 10 |
| 1234ze | | 40 | 40 | 40 |
| Total | | 100 | 100 | 100 |
| Emulsion Stability | | Stable | Stable | Stable |
| Foam Form | | Ice cream | Ice cream | Ice cream |
| | | foam | foam | foam |
| Total Oil phase, wt % of | | 62.9 | 67.9 | 72.9 |
| Formulation | | | | |
| Total Emulsifier, wt % of | | 1.9 | 1.9 | 1.9 |
| Formulation | | | | |
| dotal non-ionic emulsifiers, | | 0.9 | 0.9 | 0.9 |
| wt % of Formulation | | | | |
| HLB of non-ionic emulsifiers | | 9.56 | 9.56 | 9.56 |
| in Formulation | | | | |
| Wt % of Non-ionic emulsifier, | | 47.4% | 47.4% | 47.4% |
| based on total emulsifier | | | | |
| Ratio Cationic:NonIonic | | 1.11 | 1.11 | 1.11 |
| Emulsifier | | | | |
| Total oil phase thickener | | 4 | 4 | 4 |
| (Fatty Acid Alcohol), wt % | | | | |
| Formulation | | | | |
| Total oil phase (excluding | | 7.9 | 7.9 | 7.9 |
| 1233zd and 1234ze) | | | | |
| Ratio of solid non-ionic | | 0.225 | 0.225 | 0.225 |
| emulsifier to Total oil phase | | | | |
| thickener (Fatty Acid Alcohol) | | | | |
| Ratio of solid non-ionic | | 0.014 | 0.013 | 0.012 |
| emulsifier to Total Oil phase | | | | |
| Ratio of sold non-ionic | | 0.11 | 0.11 | 0.11 |
| emulsifier to total oil phase | | | | |
| excluding 1233zd and 1234ze | | | | |
| Total solids in oil phase | | 5.9 | 5.9 | 5.9 |
| Ratio of all solids to total | | 0.75 | 0.75 | 0.75 |
| oil phase (excluding 1233zd | | | | |
| and 1234ze) | | | | |
| Total oil phase liquids | | 2 | 2 | 2 |
| (excluding 1233zd and 1234ze) | | | | |
| Ratio of oil phase thickener | | 2 | 2 | 2 |
| to oil phase liquids | | | | |
| (excluding 1233zd and 1234zd) | | | | |
| Ratio of water based thickener | | 0 | 0 | 0 |
| to oil based thickener | | | | |
| Ratio of oil phase liquids | | 0.34 | 0.34 | 0.34 |
| (excluding 1233zd and 1234zd) | | | | |
| to all oil phase solids | | | | |
| 1233zd(E)/1234ze(E) wt ratio | | 1:4 | 1:4 | 1:4 |

Each of the formulations 1PD, 1PE and 1PF produce a stable oil-in-water emulsion and produce ice cream foam. It is noted that in each Formulation had the following characteristics:

Total of 1233zd plus 1234ze>40 wt % of the formulation
Ratio of 1233zd:1234ze=from 1:8 to 1:2
Net HLB of Solid Non-Ionic Emulsifier within Range—7-18
Total Oil Content (exclusive of 1233zd and 1234zd)—7 wt % or higher
Weight Ratio of Solid Non-Ionic Emulsifier:Total Oil Phase—(ex 1233zd+1234ze)-<0.14.
Weight Ratio of Oil Phase Thickener to all solids in oil phase (ex 1233zd+1234ze)->0.57.
Weight Ratio of Oil Phase Thickener to all liquids in oil phase (ex 1233zd+1234ze)->1.25 and about 3 or less
Weight Ratio of Oil Phase liquids (excluding 1233zzd and 1234ze) to all solids in oil phase<0.45.
Weight Ratio of Water Phase Thickener to oil phase thickener<0.02.

### Example 2—Ice Cream Foam

A personal care formulation generally as described in Table 2 (Formulation 2) below was formed:

TABLE 2

| | | HLB of | |
|---|---|---|---|
| | | non-ionic | Wt % Component |
| Ingredient | Function | emulsifier | Formulation 2 |
| water | Solvent | | 42.75 |
| Glycerin (water soluble) | humectant | | 0.5 |
| Behentrimonium Chloride | Solid Cationic | | 0 |
| (BTAC DPG 70) | emulsifier | | |
| Steareth 21 | Solid Non-ionic | 15.55 | 0.5 |
| | emulsifier | | |
| Cetearyl Alcohol (Lanette O) | fatty alcohol | | 4 |
| and/or Cetyl Alcohol (Lanette | | | |
| 16) − HLB = 15.5 | | | |
| Carnuba Wax | Wax | | 0 |
| Ester oil | Skin softener | | 1 |
| Glyceryl Stearate | non-ionic Emulsifier | 3.8 | 0 |
| | (solid) | | |
| Glycol Distearate | non-ionic Emulsifier | 5.5 | 0 |
| | (solid) | | |
| Silicone Oil | | | 1 |
| Water Based Thickner | Conditioning | | 0 |
| (Carbopol 940) | agent | | |
| TEA | pH adjuster | | 0.125 |
| Euxyl K940 | Preservative | | 0.125 |
| Fragrance (242091 GB BLDELI) | Fragrance | | 0 |
| Colorant (Red 4/Yellow 6) | Red 4/Yellow 6 | | 0 |
| 1233zd | | | 10 |
| 1234ze | | | 40 |
| Total | | | 100 |
| Total Oil Phase | | | 60.5 |
| Total Water Phase | | | 39.5 |
| | | | 100 |
| Total Emulsifier | | | 0.5 |
| Total Non-ionic emulsifier, | | | 1 |
| wt % | | | |
| Wt % Non-ionic based on total | | | 100% |
| emulsifier | | | |
| HLB of Non-ionic Emulsifiers | | | 15.55 |
| in Formulation | | | |
| Ratio of Cationic:NonIonic | | | 0.00 |
| Total Fatty Alcohol | | | 4 |
| Total Fatty Alcohol + Carnuba | | | 4 |
| Wax | | | |
| Total Oil (x of 1233zd and | | | 6.5 |
| 1234ze) | | | |
| Ratio of sold non-ionic | | | 0.25 |
| emulsifier to Thickner | | | |
| Ratio of sold non-ionic | | | 0.009 |
| emulsifier to total Oil phase | | | |
| Ratio of sold non-ionic | | | 0.077 |
| emulsifier to total Oil phase | | | |
| (excluding 1233zd and 1234ze) | | | |
| Ratio of oil phase thickener | | | 2 |
| to oil phase liquids | | | |
| (excluding 1233zd and 1234zd) | | | |
| Total Solids in Oil Phase | | | 4.5 |
| Ratio of oil phase liquids | | | 0.44 |
| (excluding 1233zd and 1234zd) | | | |
| to all oil phase solids | | | |

The above Formulation 2 formed a stable emulsion (after shaking for 24 hours at room temperature), and when dispensed through the nozzle from the pressurized container formed an ice cream foam as revealed in FIGS. 1A and 1B. It is noted that in each case the formulation had the following characteristics:

Total of 1233zd plus 1234ze>40 wt % of the formulation
Ratio of 1233zd:1234ze=from 1:8 to 1:2
Net HLB of Solid Non-Ionic Emulsifier within Range—6-18
Total Oil Content (exclusive of 1233zd and 1234zd)—7 wt % or higher
Weight Ratio of Solid Non-Ionic Emulsifier:Total Oil Phase—(ex 1233zd+1234ze)-<0.14.
Weight Ratio of Oil Phase Thickener to all solids in oil phase (ex 1233zd+1234ze)->0.57.
Weight Ratio of Oil Phase Thickener to all liquids in oil phase (ex 1233zd+1234ze)->1.25 and <3
Weight Ratio of Oil Phase liquids (excluding 1233zzd and 1234ze) to all solids in oil phase<0.45.
There is no water-based thickener present in the formulation.

### Examples 2A-2C—Ice Cream Foam

Three personal care formulations 2A-2C as described in Table 2A below are formed:

TABLE 2A

| | | HLB of | | | |
|---|---|---|---|---|---|
| | | non-ionic | Wt % Component | | |
| Ingredient | Function | emulsifier | 2A | 2B | 2C |
| water | Solvent | | 42.75 | 42.75 | 42.75 |
| Glycerin (water soluble) | humectant | | 0.5 | 0.5 | 0.5 |
| Behentrimonium Chloride (BTAC | Solid Cationic | | 0 | 0 | 0 |
| DPG 70) | emulsifier | | | | |
| Steareth 21 − HLB = 15.55 | Solid Non-ionic | 15.55 | 0.25 | 0.35 | 0.45 |
| | emulsifier | | | | |
| Cetearyl Alcohol (Lanette O) | fatty alcohol | | 4 | 4 | 4 |
| and/or Cetyl Alcohol (Lanette | | | | | |
| 16) − HLB = 15.5 − non-ionic fatty | | | | | |
| alcohol emulsifier | | | | | |
| Carnuba Wax | Wax | | 0 | 0 | 0 |
| Ester oil | Skin softener | | 1 | 1 | 1 |
| Glyceryl Stearate | non-ionic Emulsifier | 3.8 | 0.25 | 0.15 | 0.05 |
| | (solid) | | | | |
| Glycol Distearate | non-ionic Emulsifier | 5.5 | 0 | 0 | 0 |
| | (solid) | | | | |
| Silicone Oil | | | 1 | 1 | 1 |
| Water Based Thickner | Conditioning agent | | 0 | 0 | 0 |
| (Carbopol 940) | | | | | |
| TEA | pH adjuster | | 0.125 | 0.125 | 0.125 |
| Euxyl K940 | Preservative | | 0.125 | 0.125 | 0.125 |
| Fragrance (242091 GB BLDELI) | Fragrance | | 0 | 0 | 0 |
| Colorant (Red 4/Yellow 6) | Red 4/Yellow 6 | | 0 | 0 | 0 |
| 1233zd | | | 10 | 10 | 10 |
| 1234ze | | | 40 | 40 | 40 |
| Total | | | 100 | 100 | 100 |
| Total Oil Phase | | | 55.5 | 55.5 | 55.5 |
| Total Water Phase | | | 44.5 | 44.5 | 44.5 |
| | | | 100 | 100 | 100 |
| Total Emulsifier | | | 1 | 1 | 1 |
| Total Non-ionic emulsifier, | | | 1 | 1 | 1 |
| wt % | | | | | |
| Wt % Non-ionic based on total | | | 100% | 100% | 100% |
| emulsifier | | | | | |
| HLB of Non-ionic Emulsifiers | | | 9.09 | 12.03 | 14.38 |
| in Formulation | | | | | |
| Ratio of Cationic:NonIonic | | | 0.00 | 0.00 | 0.00 |
| Total Fatty Alcohol | | | 4 | 4 | 4 |
| Total Fatty Alcohol + | | | 4 | 4 | 4 |
| Carnuba Wax | | | | | |
| Total Oil (x of 1233zd and | | | 6.5 | 6.5 | 6.5 |
| 1234ze) | | | | | |
| Total solids in oil phase | | | 4.5 | 4.5 | 4.5 |
| Ratio of solid non-ionic | | | 0.250 | 0.250 | 0.250 |
| emulsifier to Thickner | | | | | |
| Ratio of sold non-ionic | | | 0.009 | 0.009 | 0.009 |
| emulsifier to total Oil phase | | | | | |
| Ratio of all oil phase solids | | | 0.692 | 0.692 | 0.692 |
| to total oil phase (excluding | | | | | |
| 1233zd and 1234ze) | | | | | |
| Ratio of sold non-ionic | | | 0.077 | 0.077 | 0.077 |
| emulsifier to total Oil phase | | | | | |
| (excluding 1233zd and 1234ze) | | | | | |
| Ratio of soil phase thickener | | | 2 | 2 | 2 |
| to total oil phase liquids | | | | | |
| (excluding 1233zd and 1234ze) | | | | | |
| Ratio of oil phaseliquids | | | 0.444 | 0.444 | 0.444 |
| (excluding zd and ze):all oil | | | | | |
| phase solids | | | | | |

The above formulations each form a stable emulsion (after shaking for 24 hours at room temperature), and when dispensed through the nozzle from the pressurized container form an ice cream foam as exemplified in Figures 1A, 1B and 2. It is noted that in each case the formulation has the following characteristics:

Total of 1233zd plus 1234ze>40 wt % of the formulation
Ratio of 1233zd:1234ze=from 1:8 to 1:2
Net HLB of Solid Non-Ionic Emulsifier within Range—6-18
Total Oil Content (exclusive of 1233zd and 1234zd)—7 wt % or higher
Weight Ratio of Solid Non-Ionic Emulsifier:Total Oil Phase—(ex 1233zd+1234ze)-<0.14.
Weight Ratio of Oil Phase Thickener to all solids in oil phase (ex 1233zd+1234ze)->0.57.
Weight Ratio of Oil Phase Thickener to all liquids in oil phase (ex 1233zd+1234ze)->1.25 and <3
Weight Ratio of Oil Phase liquids (excluding 1233zzd and 1234ze) to all solids in oil phase<0.45.
There is no water-based thickener present in the formulation.

### Example 3—Ice Cream Foam

A personal care formulation 3 generally as described in Table 3 below was formed:

TABLE 3

| | | HLB of | |
|---|---|---|---|
| | | non-ionic | Wt % Component |
| Ingredient | Function | emulsifier | Formulation 3 |
| water | Solvent | | 42.75 |
| Glycerin (water | humectant | | 0.5 |
| soluble) | | | |
| Behentrimonium | Solid Cationic | | 0 |
| Chloride (BTAC | emulsifier | | |
| DPG 70) | | | |
| Steareth 21 | Solid Non-ionic | 15.55 | 0.5 |
| | emulsifier | | |
| Cetearyl Alcohol | fatty alcohol | | 2 |
| (Lanette O) and/or Cetyl | | | |
| Alcohol (Lanette 16) − | | | |
| HLB = 15.5 | | | |
| Carnuba Wax | Wax | | 2 |
| Ester oil | Skin softener | | 1 |
| Glyceryl Stearate | non-ionic Emulsifier | 3.8 | 0 |
| | (solid) | | |
| Glycol Distearate | non-ionic Emulsifier | 5.5 | 0 |
| | (solid) | | |
| Silicone Oil | | | 1 |
| Water Based Thickner | Conditioning agent | | 0 |
| (Carbopol 940) | | | |
| TEA | pH adjuster | | 0.125 |
| Euxyl K940 | Preservative | | 0.125 |
| Fragrance (242091 GB BLDELI) | Fragrance | | 0 |
| Colorant (Red 4/Yellow 6) | Red 4/Yellow 6 | | 0 |
| 1233zd | | | 10 |
| 1234ze | | | 40 |
| Total | | | 100 |
| Total Oil Phase | | | 56.5 |
| Total Water Phase | | | 43.5 |
| | | | 100 |
| Total Emulsifier | | | 0.5 |
| Total Non-ionic emulsifier, | | | 0.5 |
| wt % | | | |
| Wt % Non-ionic based on | | | 100% |
| total emulsifier | | | |
| HLB of Non-ionic Emulsifiers | | | 15.55 |
| in Formulation | | | |
| Ratio of Cationic:NonIonic | | | 0.00 |
| Total Fatty Alcohol | | | 2 |
| Total Fatty Alcohol + | | | 4 |
| Carnuba Wax | | | |
| Total Oil (x of 1233zd and | | | 6.5 |
| 1234ze) | | | |
| Ratio of sold non-ionic | | | 0.25 |
| emulsifier to Thickener | | | |
| Ratio of sold non-ionic | | | 0.009 |
| emulsifier to total Oil | | | |
| phase | | | |
| Ratio of sold non-ionic | | | 0.077 |
| emulsifier to total Oil | | | |
| phase (excluding 1233zd | | | |
| and 1234ze) | | | |
| Ratio of oil phase thickener | | | 1 |
| to oil phase liquids | | | |
| (excluding 1233zd and | | | |
| 1234zd) | | | |
| Ratio of oil phase liquids | | | 0.444 |
| (excluding 1233zd and | | | |
| 1234zd) to all oil phase | | | |
| solids | | | |

The above Formulation 3 formed a stable emulsion (after shaking for 24 hours at room temperature), and when dispensed through the nozzle from the pressurized container formed an ice cream foam as revealed in FIG. 2. It is noted that in each case the formulation had the following characteristics:

Total of 1233zd plus 1234ze>40 wt % of the formulation
Ratio of 1233zd:1234ze=from 1:8 to 1:2
Net HLB of Solid Non-Ionic Emulsifier within Range—7-18
Total Oil Content (exclusive of 1233zd and 1234zd)—6 wt % or higher
Weight Ratio of Solid Non-Ionic Emulsifier:Total Oil Phase—(ex 1233zd+1234ze)-<0.14.
Weight Ratio of Oil Phase Thickener to all solids in oil phase (ex 1233zd+1234ze)->0.57.
Weight Ratio of Oil Phase Thickener to all liquids in oil phase (ex 1233zd+1234ze)->1.25 and <3
Weight Ratio of Oil Phase liquids (excluding 1233zzd and 1234ze) to all solids in oil phase<0.45.
There is no water-based thickener present in the formulation.

### Examples 3A-3C—Ice Cream Foam

Three personal care formulations 3A-3C as described in Table 3A below were formed:

TABLE 3A

| | | HLB of | | | |
|---|---|---|---|---|---|
| | | non-ionic | Wt % Component | | |
| Ingredient | Function | emulsifier | 3A | 3B | 3C |
| water | Solvent | | 42.75 | 35.5 | 35.5 |
| Glycerin (water soluble) | humectant | | 0.5 | 0.5 | 0.5 |
| Behentrimonium Chloride | Solid Cationic | | 0 | 0 | 0 |
| (BTAC DPG 70) | emulsifier | | | | |
| Steareth 21 − HLB = 15.55 | Solid Non-ionic | 15.55 | 0.25 | 0.35 | 0.45 |
| | emulsifier | | | | |
| Cetearyl Alcohol (Lanette O) | fatty alcohol | | 2 | 2 | 2 |
| and/or Cetyl Alcohol | | | | | |
| (Lanette 16) − HLB = 15.5 − | | | | | |
| non-ionic fatty alcohol | | | | | |
| emulsifier | | | | | |
| Carnuba Wax | Wax | | 2 | 2 | 2 |
| Ester oil | Skin softener | | 1 | 1 | 1 |
| Glyceryl Stearate | non-ionic Emulsifier | 3.8 | 0.25 | 0.15 | 0.05 |
| | (solid) | | | | |
| Glycol Distearate | non-ionic Emulsifier | 5.5 | 0 | 0 | 0 |
| | (solid) | | | | |
| Silicone Oil | | | 1 | 1 | 1 |
| Water Based Thickner | Conditioning agent | | 0 | 0 | 0 |
| (Carbopol 940) | | | | | |
| TEA | pH adjuster | | 0.125 | 0.125 | 0.125 |
| Euxyl K940 | Preservative | | 0.125 | 0.125 | 0.125 |
| Fragrance (242091 GB BLDELI) | Fragrance | | 0 | 0 | 0 |
| Colorant (Red 4/Yellow 6) | Red 4/Yellow 6 | | 0 | 0 | 0 |
| 1233zd | | | 10 | 10 | 10 |
| 1234ze | | | 40 | 40 | 40 |
| Total | | | 100 | 100 | 100 |
| Total Oil Phase | | | 55.5 | 55.5 | 55.5 |
| Total Water Phase | | | 45.5 | 45.5 | 45.5 |
| | | | 100 | 100 | 100 |
| Total Emulsifier | | | 0.5 | 0.5 | 0.5 |
| Total Non-ionic emulsifier, | | | 0.5 | 0.5 | 0.5 |
| wt % | | | | | |
| Wt % Non-ionic based on | | | 100% | 100% | 100% |
| total emulsifier | | | | | |
| HLB of Non-ionic Emulsifiers | | | 9.09 | 12.03 | 14.38 |
| in Formulation | | | | | |
| Ratio of Cationic:NonIonic | | | 0.00 | 0.00 | 0.00 |
| Total Fatty Alcohol | | | 2 | 2 | 2 |
| Total Fatty Alcohol + | | | 4 | 4 | 4 |
| Carnuba Wax | | | | | |
| Total Oil (x of 1233zd and | | | 6.5 | 6.5 | 6.5 |
| 1234ze) | | | | | |
| Ratio of sold non-ionic | | | 0.25 | 0.25 | 0.25 |
| emulsifier to Thickener | | | | | |
| Ratio of sold non-ionic | | | 0.009 | 0.009 | 0.009 |
| emulsifier to total Oil | | | | | |
| phase | | | | | |
| Ratio of sold non-ionic | | | 0.077 | 0.077 | 0.077 |
| emulsifier to total Oil | | | | | |
| phase (excluding 1233zd and | | | | | |
| 1234ze) | | | | | |
| Ratio of oil phase thickener | | | 1 | 1 | 1 |
| to total Oil phase liquids | | | | | |
| (excluding 1233zd and | | | | | |
| 1234ze) | | | | | |

Each of the above formulations form a stable emulsion (after shaking for 24 hours at room temperature), and when dispensed through the nozzle from the pressurized container form an ice cream foam as exemplified in FIG. 2. It is noted that in each case the formulation has the following characteristics:

Total of 1233zd plus 1234ze>40 wt % of the formulation
Ratio of 1233zd:1234ze=from 1:8 to 1:2
Net HLB of Solid Non-Ionic Emulsifier within Range—6-18
Total Oil Content (exclusive of 1233zd and 1234zd)—7 wt % or higher
Weight Ratio of Solid Non-Ionic Emulsifier:Total Oil Phase—(ex 1233zd+1234ze)-<0.14.
Weight Ratio of Oil Phase Thickener to all solids in oil phase (ex 1233zd+1234ze)->0.57.
Weight Ratio of Oil Phase Thickener to all liquids in oil phase (ex 1233zd+1234ze)->1.25 and <3
Weight Ratio of Oil Phase liquids (excluding 1233zzd and 1234ze) to all solids in oil phase<0.45.
There is no water-based thickener present in the formulation.

### Example 4A—Hydrating Body Lotion to Form Ice Cream Foam

A hydrating body lotion is prepared using a hydrating base comprising water, Cetearyl Alcohol (as oil phase thickener that also may contribute as a stability assistance component, as a skin softener and/or as an emollient), Glycerin (humectant), Cetyl Alcohol (phase thickener that also may contribute as a stability assistance component, as a skin softener and/or as an emollient), Behentrimonium Chloride (as a cationic emulsifier), Argania spinosa (Argan) Kernel Oil, Silk Amino Acids, Dimethicone (silicone oil), Cyclopentasiloxane (silicone oil), Dimethiconol (silicone oil), Glycol Distearate (non-ionic emulsifier HLB of 1), Glycol Stearate (stability assistance component with an HLB of 2.9), Ceteareth-20 (as an emulsifier with an HLB of 15.2), Isopropyl Alcohol, Citric Acid, Phenoxyethanol, Ethylhexylglycerin, DMDM Hydantoin, Fragrance (Parfum), Red 40 (CI 16035), Yellow 5 (CI 19140), Yellow 6 (CI 15985Water). Several formulations using this hydrating base and various amounts of trans1233zd, trans1234zd in relative amounts indicated in Table 4 below, with all amounts being in percentage by weight:

TABLE 4
Hydrating Body Lotion

| | Dosage Formulations | | | | | |
|---|---|---|---|---|---|---|
| Ingredient | A | B | C | D | E | F |
| hydrating base | 50 | 60 | 70 | 80 | 80 | 70 |
| 1233zd | 10 | 8 | 6 | 4 | | |
| 1234ze | 40 | 32 | 24 | 16 | 20 | 30 |
| Total | 100 | 100 | 100 | 100 | 100 | 100 |
| Wt ratio | 1:4 | 1:4 | 1:4 | 1:4 | NA | NA |
| 1233zd/1234ze | | | | | | |

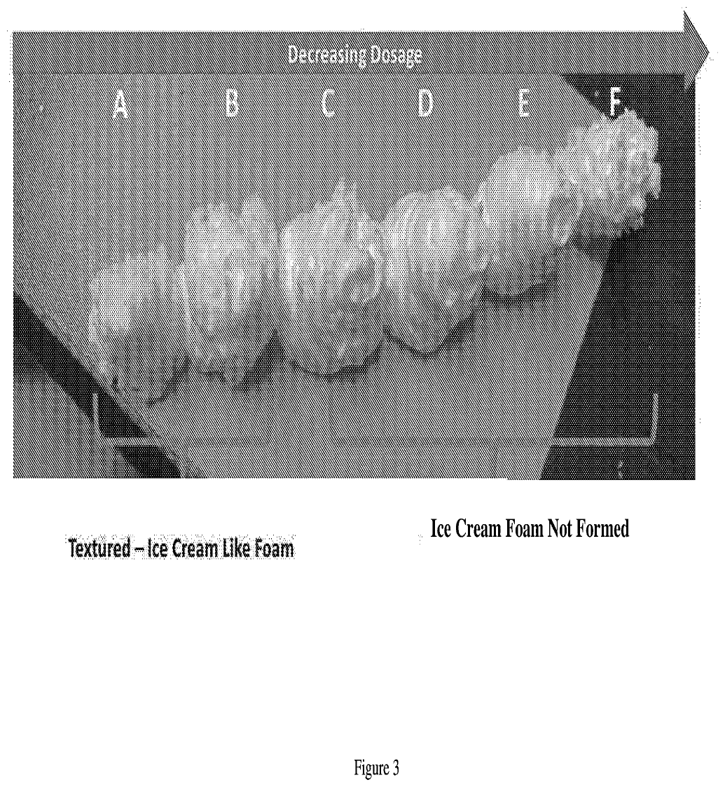

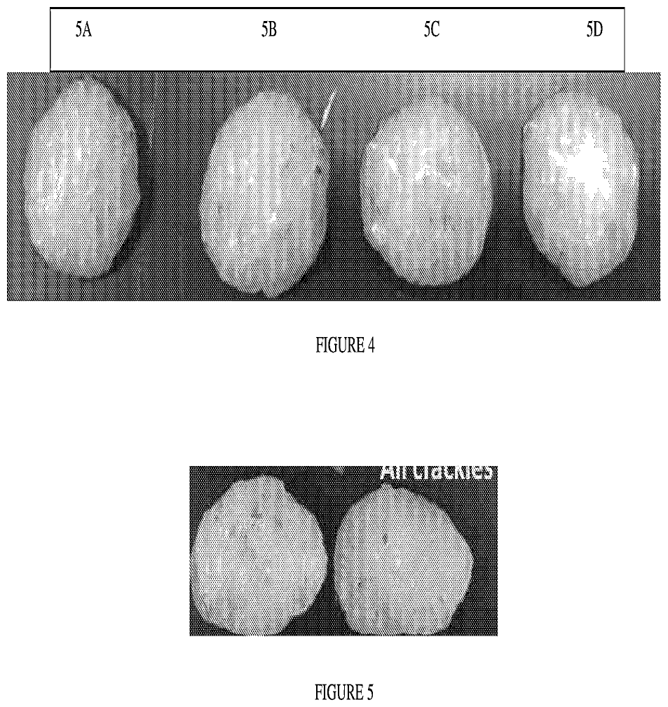

As can be seen from FIG. 3, Formulations 4A and 4B, which each utilized a total amount of trans1233zd plus trans1234ze of 40% or greater and a zd/ze weight ratio of 1:4 formed textured, ice-cream like foam, which had an opaque character. In contrast, Formulations 4C and 4D produced a foam with a relatively smooth continuous surface—ice cream foam was not produced. In these cases, even though the zd/ze weight ratio was 1:4, the total dosage was less than 40% (30% and 20%, respectively). In the case of Formulations 4E and 4F, no 1233zd was used and ice cream foam was not formed.

### Examples 4AA and 4BA—Hydrating Body Lotion to Form Ice Cream Foam

Example 4A is repeated except that zd/ze weight ratio for Formulations 4A and 4B is adjusted to 1:2 (i.e., 16.67 wt % 1233zd and 33.33 wt % 1234ze for Formulation A and 13.3 wt % 1233zd and 26.7 wt % 1234ze for formulation B). Similar results are obtained.

### Example 4AB and 4BB—Hydrating Body Lotion to Form Ice Cream Foam

Example 4 is repeated except that zd/ze weight ratio for Formulations 4A and 4B is adjusted to 1:8 (i.e., 6.25 wt % 1233zd and 43.33 wt % 1234ze for Formulation A and 5 wt % 1233zd and 45 wt % 1234ze for formulation B). Similar results are obtained.

### Example 5—Hydrating Body Lotion to Form Crackling Sherbet Foam Using Liquid Emulsifier

A series of hydrating body lotion formulations 5A through 5D were prepared as indicated in Table 5 below.

TABLE 5

| | | HLB of | | | | |
|---|---|---|---|---|---|---|
| | | non-ionic | Wt % Component | | | |
| Ingredient | Function | emulsifier | 5A | 5B | 5C | 5D |
| water | Solvent | | 47.25 | 47.25 | 47.25 | 47.25 |
| Glycerin (water soluble) | humectant | | 1 | 1 | 1 | 1 |
| Tween 20 | Liquid non-ionic | 16.7 | 0.4 | 0.65 | 0.8 | 1 |
| | emulsifier | | | | | |
| Brij 93 | Liquid non-ionic | 4 | 0.45 | 0.35 | 0.2 | 0 |
| | emulsifier | | | | | |
| Cetearyl Alcohol (Lanette O) and/or | fatty alcohol | | 0 | 0 | 0 | 0 |
| Cetyl Alcohol (Lanette 16) − HLB = | | | | | | |
| 15.5 − non-ionic fatty alcohol | | | | | | |
| emulsifier | | | | | | |
| Carnuba Wax | Wax | | 0 | 0 | 0 | 0 |
| Ester oil | Skin softener | | 0 | 0 | 0 | 0 |
| Glyceryl Stearate | non-ionic | 3.8 | 0 | 0 | 0 | 0 |
| | Emulsifier | | | | | |
| | (solid) | | | | | |
| Glycol Distearate | non-ionic Emulsifier | 5.5 | 0 | 0 | 0 | 0 |
| | (solid) | | | | | |
| Silicone Oil | | | 0 | 0 | 0 | 0 |
| Water Based Thickner | Conditioning | | 0.25 | 0.25 | 0.25 | 0.25 |
| (Carbopol 940) | agent | | | | | |
| TEA | pH adjuster | | 0.25 | 0.25 | 0.25 | 0.25 |
| Euxyl K940 | Preservative | | 0.25 | 0.25 | 0.25 | 0.25 |
| Fragrance (242091 GB BLDELI) | Fragrance | | 0 | 0 | 0 | 0 |
| Colorant (Red 4/Yellow 6) | Red 4/Yellow 6 | | 0 | 0 | 0 | 0 |
| 1233zd | | | 40 | 40 | 40 | 40 |
| 1234ze | | | 10 | 10 | 10 | 10 |
| Total | | | 100 | 100 | 100 | 100 |
| Total Oil Phase | | | 51 | 51 | 51 | 51 |
| Total Water Phase | | | 49 | 49 | 49 | 49 |
| | | | 100 | 100 | 100 | 100 |
| Total Emulsifier | | | 1 | 1 | 1 | 1 |
| Total Non-ionic emulsifier, wt % | | | 1 | 1 | 1 | 1 |
| Wt % Non-ionic based on total | | | 100% | 100% | 100% | 100% |
| emulsifier | | | | | | |
| HLB of Non-ionic Emulsifiers in | | | 9.08 | 12.26 | 14.16 | 16.7 |
| Formulation | | | | | | |
| Ratio of Cationic:NonIonic | | | 0.00 | 0.00 | 0.00 | 0.00 |
| Total Fatty Alcohol | | | 0 | 0 | 0 | 0 |
| Total Fatty Alcohol + Carnuba Wax | | | 0 | 0 | 0 | 0 |
| Total Oil (x of 1233zd and 1234ze) | | | 1 | 1 | 1 | 1 |
| Ratio of liquid non-ionic | | | 0.020 | 0.020 | 0.020 | 0.020 |
| emulsifier to Toatl Oil Phase | | | | | | |
| Ratio of liquid non-ionic | | | 1 | 1 | 1 | 1 |
| emulsifier to total Oil phase | | | | | | |
| (excluding 1233zd and 1234ze) | | | | | | |

The above formulations each formed a stable emulsion (after shaking for 24 hours at room temperature), and when dispensed through the nozzle from the pressurized container formed a crackling sherbet foam as shown in FIG. 4. It is noted that in each case the formulation has the following characteristics:

Total of 1233zd plus 1234ze>40 wt % of the formulation
Ratio of 1233zd:1234ze=from 4:1 to 1:1
Net HLB of Liquid Non-Ionic Emulsifier within Range—7-18

### Example 6—Hydrating Body Lotion to Form Crackling Sherbet Foam Using Solid Emulsifier and Ester Oil

Two hydrating body lotion formulations 6A and 6B were prepared as indicated in Table 6 below.

TABLE 6

| | | HLB of | | |
|---|---|---|---|---|
| | | non-ionic | Wt % Component | |
| Ingredient | Function | emulsifier | 6A | 6B |
| water | Solvent | | 45.25 | 45.25 |
| Glycerin (water soluble) | humectant | | 1 | 1 |
| Steareth 20 | Solid non-ionic | 16.7 | 0.45 | 0.9 |
| | emulsifier | | | |
| Brij 93 | Liquid non-ionic | 4 | 0 | 0 |
| | emulsifier | | | |
| Cetearyl Alcohol (Lanette O) | fatty alcohol | | 0 | 0 |
| and/or Cetyl Alcohol | | | | |
| (Lanette 16) − HLB = 15.5 − | | | | |
| non-ionic fatty alcohol | | | | |
| emulsifier | | | | |
| Carnuba Wax | Wax | | 0 | 0 |
| Ester oil | Skin softener | | 2 | 2 |
| Glyceryl Stearate | non-ionic Emulsifier | 3.8 | 0.55 | 0.1 |
| | (solid) | | | |
| Glycol Distearate | non-ionic Emulsifier | 5.5 | 0 | 0 |
| | (solid) | | | |
| Silicone Oil | | | 0 | 0 |
| Water Based Thickner | Conditioning agent | | 0.25 | 0.25 |
| (Carbopol 940) | | | | |
| TEA | pH adjuster | | 0.25 | 0.25 |
| Euxyl K940 | Preservative | | 0.25 | 0.25 |
| Fragrance (242091 GB BLDELI) | Fragrance | | 0 | 0 |
| Colorant (Red 4/Yellow 6) | Red 4/Yellow 6 | | 0 | 0 |
| 1233zd | | | 40 | 40 |
| 1234ze | | | 10 | 10 |
| Total | | | 100 | 100 |
| Total Oil Phase | | | 53 | 53 |
| Total Water Phase | | | 47 | 47 |
| | | | 100 | 100 |
| Total Emulsifier | | | 1 | 1 |
| Total Non-ionic emulsifier, | | | 1 | 1 |
| wt % | | | | |
| Wt % Non-ionic based on | | | 100% | 100% |
| total emulsifier | | | | |
| HLB of Non-ionic Emulsifiers | | | 9.07 | 14.33 |
| in Formulation | | | | |
| Ratio of Cationic:NonIonic | | | 0.00 | 0.00 |
| Total Fatty Alcohol | | | 0 | 0 |
| Total Fatty Alcohol + | | | 0 | 0 |
| Carnuba Wax | | | | |
| Total Oil (x of 1233zd and | | | 3 | 3 |
| 1234ze) | | | | |
| Ratio of solid non-ionic | | | 0.019 | 0.019 |
| emulsifier to Toatl Oil | | | | |
| Phase | | | | |
| Ratio of solid non-ionic | | | 0.333 | 0.333 |
| emulsifier to total Oil | | | | |
| phase (excluding 1233zd and | | | | |
| 1234ze) | | | | |

The above formulations each formed a stable emulsion (after shaking for 24 hours at room temperature), and when dispensed through the nozzle from the pressurized container formed a crackling sherbret foam, as illustrated in FIG. 5. It is noted that in each case the formulation has the following characteristics:

Total of 1233zd plus 1234ze>40 wt % of the formulation
Ratio of 1233zd:1234ze from 4:1 to 1:1
Net HLB of Liquid Non-Ionic Emulsifier within Range—7-18
Total Oil Content (exclusive of 1233zd and 1234zd)—3 wt % or higher

### Example 7—Hydrating Body Lotion to Form Crackling Sherbet Foam Using Solid Emulsifier and Oil Phase Thickener

A hydrating body lotion formulation 6 was prepared as indicated in Table 7 below.

TABLE 7

| | | HLB of | |
|---|---|---|---|
| | | non-ionic | Wt % Component |
| Ingredient | Function | emulsifier | Formulation 7 |
| water | Solvent | | 45.25 |
| Glycerin (water soluble) | humectant | | 1 |
| Steareth 21 | Solid non-ionic | 16.7 | 0.9 |
| | emulsifier | | |
| Brij 93 | Liquid non-ionic | 4 | 0 |
| | emulsifier | | |
| Cetearyl Alcohol (Lanette O) | fatty alcohol | | 2 |
| and/or Cetyl Alcohol | | | |
| (Lanette 16) − HLB = 15.5 − | | | |
| non-ionic fatty alcohol | | | |
| emulsifier | | | |
| Carnuba Wax | Wax | | 0 |
| Ester oil | Skin softener | | 0 |
| Glyceryl Stearate | non-ionic Emulsifier | 3.8 | 0.1 |
| | (solid) | | |
| Glycol Distearate | non-ionic Emulsifier | 5.5 | 0 |
| | (solid) | | |
| Silicone Oil | | | 0 |
| Water Based Thickner | Conditioning agent | | 0.25 |
| (Carbopol 940) | | | |
| TEA | pH adjuster | | 0.25 |
| Euxyl K940 | Preservative | | 0.25 |
| Fragrance (242091 GB BLDELI) | Fragrance | | 0 |
| Colorant (Red 4/Yellow 6) | Red 4/Yellow 6 | | 0 |
| 1233zd | | | 40 |
| 1234ze | | | 10 |
| Total | | | 100 |
| Total Oil Phase | | | 53 |
| Total Water Phase | | | 47 |
| | | | 100 |
| Total Emulsifier | | | 1 |
| Total Non-ionic emulsifier, | | | 1 |
| wt % | | | |
| Wt % Non-ionic based on | | | 100% |
| total emulsifier | | | |
| HLB of Non-ionic Emulsifiers | | | 14.33 |
| in Formulation | | | |
| Ratio of Cationic:NonIonic | | | 0.00 |
| Total Fatty Alcohol | | | 2 |
| Total Fatty Alcohol + | | | 2 |
| Carnuba Wax | | | |
| Total Oil (x of 1233zd and | | | 3 |
| 1234ze) | | | |
| Ratio of solid non-ionic | | | 0.019 |
| emulsifier to Toatl Oil | | | |
| Phase | | | |
| Ratio of solid non-ionic | | | 0.333 |
| emulsifier to total Oil | | | |
| phase (excluding 1233zd and | | | |
| 1234ze) | | | |

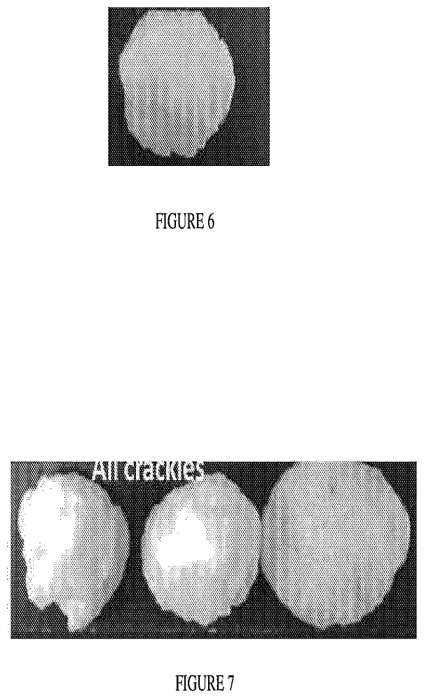

The above formulation formed a stable emulsion (after shaking for 24 hours at room temperature), and when dispensed through the nozzle from the pressurized container formed a crackling sherbret foam, as illustrated in FIG. 6. It is noted that the formulation had the following characteristics:

Total of 1233zd plus 1234ze>40 wt % of the formulation
Ratio of 1233zd:1234ze=from 4:1 to 1:1
Net HLB of Liquid Non-Ionic Emulsifier within Range—7-18
Total Oil Content (exclusive of 1233zd and 1234zd)—3 wt % or higher

### Example 8—Hydrating Body Lotion to Form Crackling Sherbet Foam Using Liquid Emulsifier, Oil Phase Thickener and Ester Oil

Three hydrating body lotion formulations 8A-8C were prepared as indicated in Table 8 below.

TABLE 8

| | | HLB of | Wt % Component | | |
|---|---|---|---|---|---|
| | | non-ionic | Formulation | Formulation | Formulation |
| Ingredient | Function | emulsifier | 8A | 8B | 8C |
| water | Solvent | | 39.25 | 41.25 | 41.25 |
| Glycerin (water soluble) | humectant | | 1 | 1 | 1 |
| Tween 20 | Liquid non-ionic | 16.7 | 0.8 | 0.8 | 0.8 |
| | emulsifier | | | | |
| Brij 93 | Liquid non-ionic | 4 | 0.2 | 0.2 | 0.2 |
| | emulsifier | | | | |
| Cetearyl Alcohol (Lanette O) | fatty alcohol | | 12 | 8 | 4 |
| and/or Cetyl Alcohol | | | | | |
| (Lanette 16) − HLB = 15.5 − | | | | | |
| non-ionic fatty alcohol | | | | | |
| emulsifier | | | | | |
| Carnuba Wax | Wax | | 0 | 0 | 0 |
| Ester oil | Skin softener | | 0 | 0 | 0 |
| Glyceryl Stearate | non-ionic Emulsifier | 3.8 | 0 | 0 | 0 |
| | (solid) | | | | |
| Glycol Distearate | non-ionic Emulsifier | 5.5 | 0 | 0 | 0 |
| | (solid) | | | | |
| Silicone Oil | | | 0 | 0 | 0 |
| Water Based Thickner | Conditioning agent | | 0.25 | 0.25 | 0.25 |
| (Carbopol 940) | | | | | |
| TEA | pH adjuster | | 0.25 | 0.25 | 0.25 |
| Euxyl K940 | Preservative | | 0.25 | 0.25 | 0.25 |
| Fragrance (242091 GB BLDELI) | Fragrance | | 0 | 0 | 0 |
| Colorant (Red 4/Yellow 6) | Red 4/Yellow 6 | | 0 | 0 | 0 |
| 1233zd | | | 40 | 40 | 40 |
| 1234ze | | | 10 | 10 | 10 |
| Total | | | 100 | 100 | 100 |
| Total Oil Phase | | | 59 | 57 | 59 |
| Total Water Phase | | | 41 | 37 | 41 |
| | | | 100 | 100 | 100 |
| Total Emulsifier | | | 1 | 1 | 1 |
| Total Non-ionic emulsifier, | | | 1 | 1 | 1 |
| wt % | | | | | |
| Wt % Non-ionic based on | | | 100.00% | 100.00% | 100.00% |
| total emulsifier | | | | | |
| HLB of Non-ionic Emulsifiers | | | 14.16 | 14.16 | 14.16 |
| in Formulation | | | | | |
| Ratio of Cationic:NonIonic | | | | | |
| Total Fatty Alcohol | | | 6 | 4 | 2 |
| Total Fatty Alcohol + | | | 6 | 4 | 2 |
| Carnuba Wax | | | | | |
| Total Oil (x of 1233zd and | | | 9 | 7 | 5 |
| 1234ze) | | | | | |
| Ratio of liquid non-ionic | | | 0.017 | 0.018 | 0.018 |
| emulsifier to Toatl Oil | | | | | |
| Phase | | | | | |
| Ratio of liquid non-ionic | | | 0.111 | 0.143 | 0.2 |
| emulsifier to total Oil | | | | | |
| phase (excluding 1233zd and | | | | | |
| 1234ze) | | | | | |

The above formulations each formed a stable emulsion (after shaking for 24 hours at room temperature), and when dispensed through the nozzle from the pressurized container formed a crackling sherbret foam, as illustrated in FIG. 7. It is noted that the formulation had the following characteristics:

Total of 1233zd plus 1234ze>40 wt % of the formulation
Ratio of 1233zd:1234ze=from 4:1 to 1:1
Net HLB of Liquid Non-Ionic Emulsifier within Range—7-18
Total Oil Content (exclusive of 1233zd and 1234zd)—3 wt % or higher

### Example 9—Hydrating Body Lotion to Form Crackling Sherbet Foam Using Solid Emulsifier, Oil Phase Thickener and Ester Oil

A hydrating body lotion formulation 9 was prepared as indicated in Table 9 below.

TABLE 9

| | | HLB of | |
|---|---|---|---|
| | | non-ionic | Wt % Component |
| Ingredient | Function | emulsifier | Formulation 9 |
| water | Solvent | | 43.25 |
| Glycerin (water soluble) | humectant | | 1 |
| Steareth 21 | Solid non-ionic | 16.7 | 0.8 |
| | emulsifier | | |
| Brij 93 | Liquid non-ionic | 4 | 0 |
| | emulsifier | | |
| Cetearyl Alcohol (Lanette O) | fatty alcohol | | 2 |
| and/or Cetyl Alcohol | | | |
| (Lanette 16) − HLB = 15.5 − | | | |
| non-ionic fatty alcohol | | | |
| emulsifier | | | |
| Carnuba Wax | Wax | | 0 |
| Ester oil | Skin softener | | 2 |
| Glyceryl Stearate | non-ionic Emulsifier | 3.8 | 0.2 |
| | (solid) | | |
| Glycol Distearate | non-ionic Emulsifier | 5.5 | 0 |
| | (solid) | | |
| Silicone Oil | | | 0 |
| Water Based Thickner | Conditioning agent | | 0.25 |
| (Carbopol 940) | | | |
| TEA | pH adjuster | | 0.25 |
| Euxyl K940 | Preservative | | 0.25 |
| Fragrance (242091 GB BLDELI) | Fragrance | | 0 |
| Colorant (Red 4/Yellow 6) | Red 4/Yellow 6 | | 0 |
| 1233zd | | | 40 |
| 1234ze | | | 10 |
| Total | | | 100 |
| Total Oil Phase | | | 55 |
| Total Water Phase | | | 45 |
| | | | 100 |
| Total Emulsifier | | | 1 |
| Total Non-ionic emulsifier, | | | 1 |
| wt % | | | |
| Wt % Non-ionic based on | | | 100.00% |
| total emulsifier | | | |
| HLB of Non-ionic Emulsifiers | | | 14.16 |
| in Formulation | | | |
| Ratio of Cationic:NonIonic | | | 0.00 |
| Total Fatty Alcohol | | | 2 |
| Total Fatty Alcohol + | | | 2 |
| Carnuba Wax | | | |
| Total Oil (x of 1233zd and | | | 5 |
| 1234ze) | | | |
| Ratio of solid non-ionic | | | 0.018 |
| emulsifier to Total Oil | | | |
| Phase | | | |
| Ratio of solid non-ionic | | | 0.2 |
| emulsifier to total Oil | | | |
| phase (excluding | | | |
| 1233zd and 1234ze) | | | |

The above formulations each formed a stable emulsion (after shaking for 24 hours at room temperature), and when dispensed through the nozzle from the pressurized container formed a crackling sherbret foam, as illustrated in FIG. 8, that had a pleasant and subtle crackling sound. It is noted that the formulation had the following characteristics:

Total of 1233zd plus 1234ze>40 wt % of the formulation
Ratio of 1233zd:1234ze from 4:1 to 1:1
Net HLB of Liquid Non-Ionic Emulsifier within Range—7-18
Total Oil Content (exclusive of 1233zd and 1234zd)—3 wt % or higher

### Example 10—Hydrating Body Lotion to Form Crackling Sherbet Foam Using Solid Emulsifier, Oil Phase Thickener, Ester Oil and Silicone Oil

A hydrating body lotion formulation 10 was prepared as indicated in Table 10 below.

TABLE 10

| | | HLB of | |
|---|---|---|---|
| | | non-ionic | Wt % Component |
| Ingredient | Function | emulsifier | Formulation 10 |
| water | Solvent | | 42.75 |
| Glycerin (water soluble) | humectant | | 1 |
| Steareth 21 | Solid non-ionic | 16.7 | 0.8 |
| | emulsifier | | |
| Brij 93 | Liquid non-ionic | 4 | 0 |
| | emulsifier | | |
| Cetearyl Alcohol | fatty alcohol | | 3 |
| (Lanette O) and/or Cetyl | | | |
| Alcohol (Lanette 16) − HLB = | | | |
| 15.5 − non-ionic fatty | | | |
| alcohol emulsifier | | | |
| Carnuba Wax | Wax | | 0 |
| Ester oil | Skin softener | | 0 |
| Glyceryl Stearate | non-ionic Emulsifier | 3.8 | 0.2 |
| | (solid) | | |
| Glycol Distearate | non-ionic Emulsifier | 5.5 | 0 |
| | (solid) | | |
| Silicone Oil | | | 1.5 |
| Water Based Thickner | Conditioning agent | | 0.125 |
| (Carbopol 940) | | | |
| TEA | pH adjuster | | 0.25 |
| Euxyl K940 | Preservative | | 0.25 |
| Fragrance (242091 GB BLDELI) | Fragrance | | 0 |
| Colorant (Red 4/Yellow 6) | Red 4/Yellow 6 | | 0 |
| 1233zd | | | 40 |
| 1234ze | | | 10 |
| Total | | | 100 |
| Total Oil Phase | | | 60 |
| Total Water Phase | | | 40 |
| | | | 100 |
| Total Emulsifier | | | 1 |
| Total Non-ionic emulsifier, | | | 1 |
| wt % | | | |
| Wt % Non-ionic based on | | | 100.00% |
| total emulsifier | | | |
| HLB of Non-ionic Emulsifiers | | | 14.16 |
| in Formulation | | | |
| Ratio of Cationic:NonIonic | | | 0.00 |
| Total Fatty Alcohol | | | 3 |
| Total Fatty Alcohol + | | | 3 |
| Carnuba Wax | | | |
| Total Oil (x of 1233zd and | | | 5.5 |
| 1234ze) | | | |
| Ratio of liquid non-ionic | | | 0.018 |
| emulsifier to Toatl Oil | | | |
| Phase | | | |
| Ratio of solid non-ionic | | | 0.18 |
| emulsifier to total Oil | | | |
| phase (excluding 1233rd and | | | |
| 1234ze) | | | |

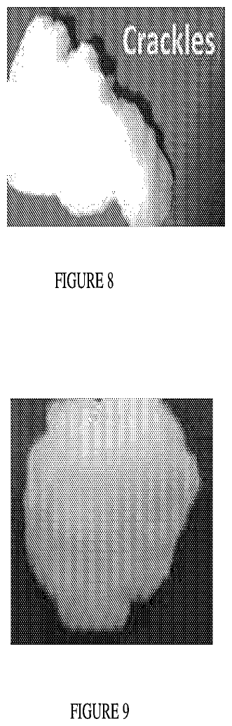

The above formulations each formed a stable emulsion (after shaking for 24 hours at room temperature), and when dispensed through the nozzle from the pressurized container formed a crackling sherbret foam, as illustrated in FIG. 9, that had a pleasant and subtle crackling sound. It is noted that the formulation had the following characteristics:

Total of 1233zd plus 1234ze>40 wt % of the formulation
Ratio of 1233zd:1234ze=from 4:1 to 1:1
Net HLB of Liquid Non-Ionic Emulsifier within Range—7-18
Total Oil Content (exclusive of 1233zd and 1234zd)—3 wt % or higher.

### Example 11—Hydrating Body Lotion to Form Crackling Sherbet Foam Using Solid Emulsifier, Oil Phase Thickener, Ester Oil and Silicone Oil

A hydrating body lotion is prepared using a hydrating base comprising water, Cetearyl Alcohol (as oil phase thickener that also may contribute as a stability assistance component, as a skin softener and/or as an emollient), Glycerin (humectant), Cetyl Alcohol (il phase thickener that also may contribute as a stability assistance component, as a skin softener and/or as an emollient), Behentrimonium Chloride (as a cationic emulsifier), Argania spinosa (Argan) Kernel Oil, Silk Amino Acids, Dimethicone (silicone oil), Cyclopentasiloxane (silicone oil), Dimethiconol (silicone oil), Glycol Distearate (non-ionic emulsifier HLB of 1), Glycol Stearate (stability assistance component with an HLB of 2.9), Ceteareth-20 (as an emulsifier with an HLB of 15.2), Isopropyl Alcohol, Citric Acid, Phenoxyethanol, Ethylhexylglycerin, DMDM Hydantoin, Fragrance (Parfum), Red 40 (CI 16035), Yellow 5 (CI 19140), Yellow 6 (CI 15985Water). Several formulations using this hydrating base and various amounts of trans1233zd, trans1234zd in relative amounts indicated in Table 11 below, with all amounts being in percentage by weight:

TABLE 11
Hydrating Body Lotion

Dosage Formulations

| Ingredient | 11A | 11B | 11C | 11D |
|---|---|---|---|---|
| hydrating base | 50 | 60 | 70 | 80 |
| 1233zd | 40 | 32 | 24 | 16 |
| 234ze | 10 | 8 | 6 | 4 |
| Total | 100 | 100 | 100 | 100 |
| Wt ratio | 4:1 | 4:1 | 4:1 | 4:1 |
| 1233zd/1234ze | | | | |

As can be seen from FIG. 10, Formulation 11A and Formulation 11B, which each utilized a total amount of trans1233zd plus trans1234ze of 40% or greater and a zd/ze weight ratio of 4:1, formed a sherbet foam having a translucent character and a pleasant crackling sound. In contrast, Formulations 11C and 11D were stable but did not produce sherbret foam; instead, these formulations produced a foam with a relatively smooth, continuous and opaque surface. In these cases, even though the zd/ze weight ratio was 4:1 the total dosage was less than 40% (30% and 20%, respectively), and sherbret foam was not formed.

### Examples 11AA and 11BA—Hydrating Body Lotion to Form Sherbet Foam

Examples 11A and 11B are repeated except that zd/ze weight ratio for formulations 11A and 11B is adjusted to 1:1 (i.e., 25 wt % zd and 25 wt % ze for Formulation 11AA and 20 wt % zd and 20 wt % ze for formulation 11AB). Similar results are obtained.

### Example 11CA and 11DA—Hydrating Body Lotion to Form Sherbet Foam

Example 11A and 11B are repeated except that zd/ze weight ratio for formulations 11A and B is adjusted to 2:1 (i.e., 33.33t % zd and 16.67 wt % ze for Formulation 11CA and 26.7 wt % zd and 13.3 wt % ze for Formulation 11DA). Similar results are obtained.

### Example 11EA and 11FA—Hydrating Body Lotion to Form Sherbet Foam

Example 11A and 11B are repeated except that zd/ze weight ratio for formulations 11A and B is adjusted to 8:1 (i.e., 6.25 wt % zd and 43.75 wt % ze for Formulation 11EA and 26.7 wt % zd and 13.3 wt % ze for Formulation 11DA). Similar results are obtained.

Examples 1 through 11 thus reveal that that formulations in which trans1233zd and trans1234ze together comprise at least about 40% by weight of the personal care composition is preferred to form ice cream and sherbet type of foam.

Also, it is revealed that ratio of trans1233zd:trans1234ze of from about 8:1 to about 1:1, more preferably about 4:1 to about 1:1 is preferred to form a sherbet like structure which also preferably crackles upon application and/or provides cooling upon application.

### Examples 12A-12E Stable Bubbling Personal Care Formulations

A series of personal care formulations were prepared using the components and the amounts as identified in Table 12 below by first forming a concentrate of the of the ingredients in the formulation (shown under the heading Conc. in Table 12) and then preparing a full formulation by combining the concentrate in a 50:50 weight ratio with a combination of 1233zd and 1234ze, with this final formulation being identified under the headings “Final”:

TABLE 12

| | | | Wt % Component | | | | | | | | | |
|---|---|---|---|---|---|---|---|---|---|---|---|---|
| | | | 12A | | 12B | | 12C | | 12D | | 12E | |
| Phase | Ingred. | Function | con. | Total | Con | Total | Con | Total | Con | Total | Con | Total |
| W | water | Solvent | 70.05 | 35.025 | 70.15 | 35.075 | 89.25 | 44.625 | 86.85 | 43.425 | 62.13 | 31.065 |
| W | EDTA-2Na | Water stabilizer | 0.1 | 0.05 | 0.1 | 0.05 | 0.1 | 0.05 | 0.1 | 0.05 | 0.1 | 0.05 |
| W | AMP-ULTRA | pH adjuster | 0.25 | 0.125 | 0.25 | 0.125 | 0.25 | 0.125 | 0.25 | 0.125 | 0.25 | 0.125 |
| | PC 2000 | | | | | | | | | | | |
| W | 1,2-Propylene | humectant | 5 | 2.5 | 5 | 2.5 | 5 | 2.5 | 5 | 2.5 | 5 | 2.5 |
| | Glycol | | | | | | | | | | | |
| W | Natrosol ™ | Thickening agent | 0.1 | 0.05 | 0.1 | 0.05 | 0.1 | 0.05 | 0.1 | 0.05 | 0.1 | 0.05 |
| | 250HHR | | | | | | | | | | | |
| | Total Water | | 75.5 | 37.75 | 75.6 | 37.8 | 94.7 | 47.35 | 92.3 | 46.15 | 67.58 | 33.79 |
| | Phase | | | | | | | | | | | |
| O | Pemulen TR-1 | Synthetic Polymeric | 0.2 | 0.1 | 0.2 | 0.1 | 0.2 | 0.1 | 0.2 | 0.1 | 0.2 | 0.1 |
| | | emulsifier | | | | | | | | | | |
| O | Carbopol ® Ulirez 10 | Rheology modifier | 0.2 | 0.1 | 0.2 | 0.1 | 0.2 | 0.1 | 0.2 | 0.1 | 0.2 | 0.1 |
| O | TEGOSOFT | emollient and | 6 | 3 | 6 | 3 | 1.2 | 0.6 | 1.8 | 0.9 | 7.98 | 3.99 |
| | TN C12-15 | texture enhancing | | | | | | | | | | |
| | Alkyl Benzoate | agent | | | | | | | | | | |
| O | 2-EHP | emollient | 18 | 9 | 18 | 9 | 3.6 | 1.8 | 5.4 | 2.7 | 23.94 | 11.97 |
| O | Span 80 | Emulsifier (solid) | 0.1 | 0.05 | 0 | 0 | 0.1 | 0.05 | 0.1 | 0.05 | 0.1 | 0.05 |
| | (HLB 4.3) | Sorbitan Oleate | | | | | | | | | | |
| | Total Oil | | 24.5 | 12.25 | 24.4 | 12.2 | 5.3 | 2.65 | 7.7 | 3.85 | 32.42 | 16.21 |
| | (excluding zd | | | | | | | | | | | |
| | and zd) | | | | | | | | | | | |
| O | 1233zd | | | 40 | | 40 | | 40 | | 40 | | 40 |
| O | 1234ze | | | 10 | | 10 | | 10 | | 10 | | 10 |
| O | Total Oil (incl | | | 62.25 | | 62.2 | | 52.65 | | 53.85 | | 66.21 |
| | zdandzd) | | | | | | | | | | | |
| | Total Oil + Water | | 100 | 100 | 100 | 100 | 100 | 100 | 100 | 100 | 100 | 100 |
| | Phase | | | | | | | | | | | |
| | HLB of Emulsifiers | | 4.3 | | 4.3 | 4.3 | 4.3 | | 4.3 | | 4.3 | |
| | in Formulation | | | | | | | | | | | |
| WEIGHT RATIOS | | | | | | | | | | | | |
| | 1233ZD:1234ZE | | 4.00 | | | 4.00 | | 4.00 | | 4.00 | | 4.00 |
| | Oil:Water | | 0.32 | | 0.32 | | 0.06 | | 0.08 | | 0.48 | |
| | Concentrate | | | | | | | | | | | |
| | Total Oil:polymeric | | | 623 | | 622 | | 527 | | 539 | | 662 |
| | emulsifiier wt | | | | | | | | | | | |
| | ratio in final | | | | | | | | | | | |
| | Total Oil:Total | | | 415 | | 622 | | 351 | | 359 | | 441 |
| | Emmulsifier wt | | | | | | | | | | | |
| | ratio in final | | | | | | | | | | | |
| | Oil:Water Total | | | 1.65 | | 1.65 | | 1.11 | | 1.17 | 0 | 1.96 |
| PROPERTIES | | | | | | | | | | | | |
| | Phase Stability | | | Yes | | Yes | | Yes | | Yes | | Yes |
| | (Yes or No) | | | | | | | | | | | |
| | Bubbling Foam | | | Yes | | Yes | | Yes | | Yes | | Yes |
| | (Yes or No) | | | | | | | | | | | |

As can be seen from Table 12 above, the personal care formulations 12A through 12E each included the emulsification polymer Pemulen TR-1 in an amount effective to provide a stable emulsion, that is, this formulation had a weight ratio of total oil:Pemulen TR-1 greater than 515 but less than about 740, which for this formulation were sufficient to form a stable emulsion, even without the use of a high HLB emulsifier. This formulation was then used to form a personal care foam that was a bubbling foam.

### Example 13—Clear Formulations with 1233zd and DRIB

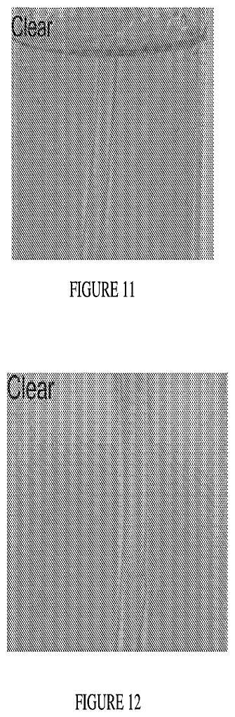

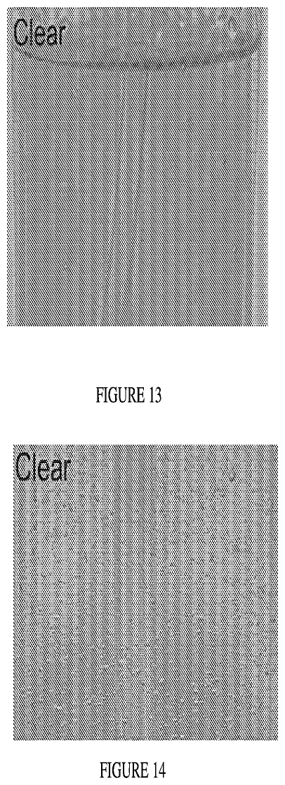

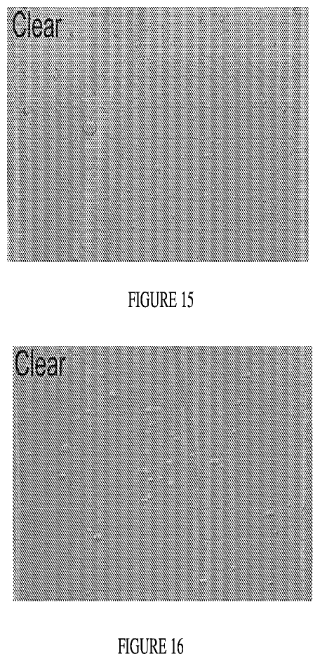

Several clear formulations are formed in accordance with the present invention as indicated in Table 13 below and as shown in FIGS. 11-14 hereof by combining the components as indicated to form the oil part and then emulsifying the oil part in water in the indicated concentration of the oil part in the formulation.

TABLE 13

| Weight Ratio of oil components | | | | | |
|---|---|---|---|---|---|
| HFO- | DRIB | | Oil | Refractive | |
| 1233zd | Cyclopenta- | Phenethyl | wt | Index of | Relevant |
| (E) | silozane (CPS) | benzoate | % | combination | FIG. |
| 5.5 | 1 | 0 | 6.5 | 1.33 | 11 |
| 20 | 0 | 1 | 21 | 1.33 | 12 |
| 6.6 | 1.2 | 0 | 7.8 | ?1.33_{——}? | 13 |
| 6.6 | 1.2 | | 15.6 | ?_{——}? | 14 |

### Example 14—Clear Formulations with 1233zd, DRIB and CRIB

Several clear formulations are formed in accordance with the present invention as indicated in Table 14 below and as shown in FIGS. 15 and 16 hereof by combining the components as indicated to form the oil part and then emulsifying the oil part in water in the indicated concentration of the oil part in the formulation.

TABLE 14

| Weight Ratio of oil | | | | | | | | |
|---|---|---|---|---|---|---|---|---|
| components | | | | | | | | |
| HFO- | | | | | | | | |
| 1233zd | | | | | | | | |
| (E) | | | Weight Ratio of | | | | | |
| HFO- | DRIB | | Water Components | | | | | |
| 1233zd | | Phenethyl | | CRIB | Oil | Refractive Index | | Relevant |
| (E) | CPS | benzoate | Water | (glycerine) | wt % | Oil | Water | Figure |
| 3 | 1 | 0 | 9 | 1 | 4 | | 1.343 | 15 |
| 9 | 0 | 1 | 9 | 1 | 10? | | 1.343 | 16 |

### Example 15—Clear Personal Care Formulation with 1233zd and DRIB

A personal care formulation (skin moisturizer) was prepared according to Table 15 below:

TABLE 15

| Component | Wt % | Function |
|---|---|---|
| Water | 69.3 | carrier |
| Xanthan gum | 0.1 | (thickner/stabilizer) |
| Simugel EG | 3 | Surfactant |
| SymSave H | 0.5 | Preservative booster |
| Hydrolite 5 | 2 | Skin moisturizer |
| Hydrovance | 2 | Moisturizing agent |
| Eucalyptus, peppermint gum1 | 0.1 | Skin health |
| 1233zd(E) | 20 | |
| Combination of cyclopentasiloxane | 3 | DRIB |
| and cyclohexasiloxane (sold as | | |
| XIAMETER PMX-0345-345) | | |

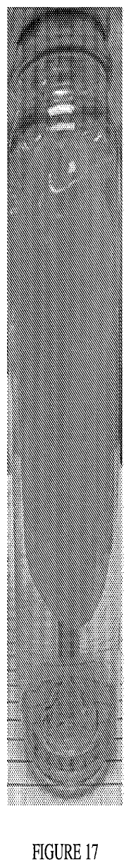

The formulation of Table 15 above was semi-transparent at room temperature as shown in the photographs provided as FIG. 17.

### Example 16—Clear Personal Care Formulation with 1233zd, DRIB and CRIB

A personal care formulation (skin moisturizer) was prepared according to Table 16 below:

TABLE 16

| Component | Wt % | Function |
|---|---|---|
| Water | | carrier |
| Simugel EG | 3 | Surfactant |
| Hydrovance | 2 | Moisturizing agent |
| Essential Oil | 0.1 | Skin health |
| 1,2 Pentylene glycol | 2 | CRIB |
| Preservative | 0.5 | |
| 1233zd(E) | 20 | |
| Combination of | 3 | DRIB |
| cyclopentasiloxane | | |
| and cyclohexasiloxane | | |
| (sold as Dowsil-345) | | |

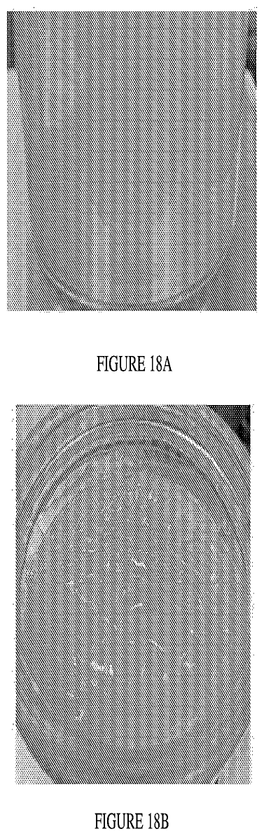

The formulation of Table 16 above was semi-transparent at room temperature and a photograph of the formulation is provided as FIG. 18A and a photograph of the foam as dispensed is provided as FIG. 18B.

### Examples 17A and 17B—Ultra Crackling Foam

Two personal care formulations (17A and 17B) are formed with concentrations of trans1234ze greater than 60% by weight and with trans1233zd:trans1234ze in a weight ratio of 7.56:1 as reported in Table 17 below were formed and sprayed from a container as described herein to form a foam. The results are reported in Table 17 below.

TABLE 17

| | | Wt % Component | |
|---|---|---|---|
| Ingredient | Function | 17A | 17BB |
| water | Solvent | 17.204 | 17.618 |
| Forlan C24 (HLB 13) | Solid non-ionic | 0.644 | 0.322 |
| polyethylene glycol ether | Emulsifier | | |
| of cholesterol and a | | | |
| polyethylene glycol ether | | | |
| of cetyl alcohol - light | | | |
| solid flake | | | |
| Natrosol ™ 250 HR | Thickner (water | 0.368 | 0.368 |
| (Hydroxyethylcellulose) | based) | | |
| Tween-20 (HLB = 16.7) | Liquid non-ionic | 0.184 | 0.092 |
| Polyethylene glycol sorbitan | Emulsifier | | |
| monolaurate, | (non-ionic | | |
| (polyoxyethylene | viscous | | |
| (20)) | liquid) | | |
| Ethanol (200 Proof) | Carrier/Solvent | 4.6 | 4.6 |
| 1233zd | | 68 | 68 |
| 1234ze | | 9 | 9 |
| Total | | 100 | 100 |
| RATIOS (weight) | | | |
| 1233zd:1234ze | | 7.56 | 7.56 |
| 1233zd:ethanol | | 14.8 | 14.7 |
| 1234ze:liquid emulsifier | | 370:1 | 739:1 |
| 1234ze:film former | | 105.6:1 | 211.2 |
| 1234ze:thickner | | 184 | 184 |
| Liquid | | 0.5 | 0.5 |
| Emulsifier:thickner | | | |
| TEST RESULTS | | | |
| Spray Quality* | | 5 | 5 |
| Foam Quality** | | 5 | 5 |
| Phase stability | | Stable | Stable |
| Cracking Loudness*** | | 5 | 5 |

*1-5 range (1 = hard to spray, 5 = easily sprayed)
**1-5 range (1 = flat foam (poor quality), 5 = good quality foam)
***1-5 range (1 = low sound, 5 = loud (ultra) crackling)

### Example 18A, 18B, 18C and 18D—Personal Care Formulation with High HLB Liquid Emulsifier to Make Cooling Foam but not Ice Cream Foam

A series of personal care formulations (18A-18D) which contained 50% by weight of trans11233zd plus trans11234ze in a weight ratio of 1:4 as depicted in Table 18 below were formed:

TABLE 18

| | | Wt % Component | | | |
|---|---|---|---|---|---|
| Ingredient | Function | 18A | 18B | 18C | 18D |
| water | Solvent | 47.5 | 47.5 | 47.5 | 47.5 |
| Glycerin | humectant | 1 | 1 | 1 | 1 |
| Carbopol 940 | Thickener | 0.25 | 0.25 | 0.25 | 0.25 |
| | (water | | | | |
| | based) | | | | |
| TEA | Neutralizing | 0.25 | 0.25 | 0.25 | 0.25 |
| | Agent | | | | |
| Euxyl K940 | Preservative | 0.25 | 0.25 | 0.25 | 0.25 |
| Tween-20 | Emulsifier | 0.4 | 0.65 | 0.8 | 1 |
| (HLB = 16.7) | (liquid) | | | | |
| Brij 93 | Emulsifier | 0.6 | 0.35 | 0.2 | 0 |
| (HLB = 4) | (liquid) | | | | |
| 1233zd | | 10 | 10 | 10 | 10 |
| 1234ze | | 40 | 40 | 40 | 40 |
| Total | | 100 | 100 | 100 | 100 |
| HLB of | | 9.08 | 12.26 | 14.16 | 16.7 |
| Emulsifiers in | | | | | |
| Formulation | | | | | |

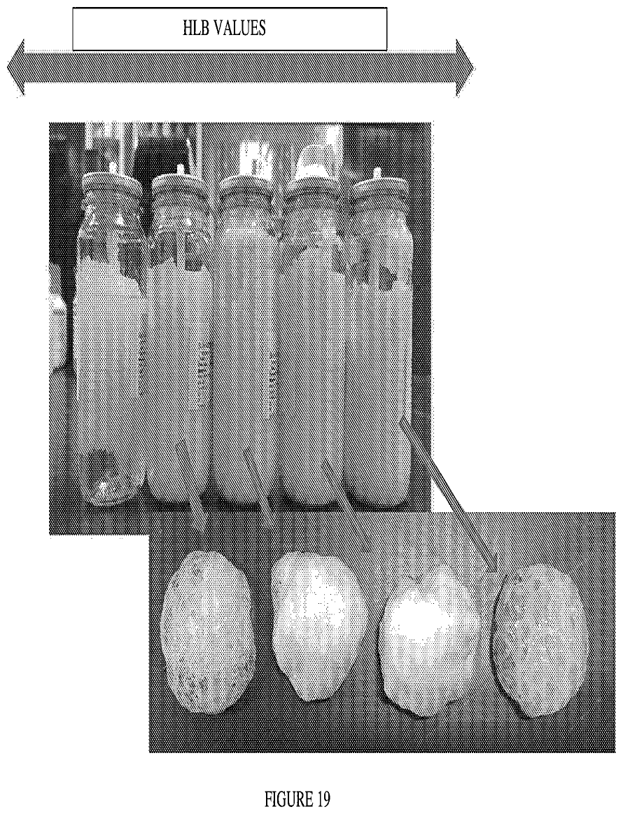

As can be seen from the table above, the series of personal care formulations had emulsifier HLB values of 9.08, 12.26, 14.16 and 16.7, for Examples 18A, 18B, 18C and 18D, respectively. However, only a liquid, non-ionic high HLB emulsifier (Tween 20) was used to increase the HLB value of the emulsification package. A stable emulsion was formed in each case, and cooling foams were formed from each formulation. These results are revealed in FIG. 19 hereof, with the photographs of the formulations in the container being from Examples 19A-19D from left to right to form the foams as indicated by the arrows in the Figure. Ice cream foam was not formed from any of the formulations.

### Example 19A and 19B—Personal Care Formulation with High HLB Solid Emulsifier and No Oil Phase Thickener—Stable Formulation; Ice Cream Foam not Formed

Two personal care formulations (19A and 19B) were formed as described in Table 19 below:

TABLE 19

Wt % Component

| Ingredient | Function | 19A | 19B |
|---|---|---|---|
| water | Solvent | 45.25 | 45.25 |
| Glycerin | humectant | 1 | 1 |
| Carbopol 904 | Thickener | 0.25 | 0.25 |
| | (water | | |
| | based) | | |
| TEA | Neutralizing | 0.25 | 0.25 |
| | Agent | | |
| Euxyl K940 | Preservative | 0.25 | 0.25 |
| Steareth-21 | Emulsifier | 0.45 | 0.9 |
| (HLB = 15.55) | (solid) | | |
| Glyceryl | Emulsifier | 0.55 | 0.1 |
| Sterarate | (solid) | | |
| Isopropyl | Stability | 2 | 2 |
| myristate | assistance | | |
| | component | | |
| 1233zd | | 10 | 10 |
| 1234ze | | 40 | 40 |
| Total | | 100 | 100 |
| HLB of | | 9.06 | 14.33 |
| Emulsifiers in | | | |
| Formulation | | | |
| Stability of the | | Stable | Stable |
| formlation | | | |

As can be seen from the table above, the addition of 2% by weigh of ester oil (isopropyl myristate) assisted with and enabled the creation of a stable personal care composition. However, no oil phase thickener was present in the formulation and ice cream foam was not formed from either of the formulations. Cooling foams were formed similar to those depicted in FIG. 17.

### Example 20—Personal Care Formulation with High HLB Solid Emulsifier and Oil Phase Thickener—Stable Formulation; Ice Cream Foam not Formed

A series of three personal care formulations (20A, 20B and 20C) as described in Table 20 below which contained 50% by weight of trans11233zd plus trans11234ze and using a high HLB, solid non-ionic emulsifier were formed.

TABLE 20

Wt % Component

| Ingredient | Function | 20A | 20B | 20C |
|---|---|---|---|---|
| water | Solvent | 43.375 | 41.375 | 39.375 |
| Glycerin | humectant | 1 | 1 | 1 |
| Carbopol 904 | Thickener | 0.125 | 0.125 | 0.125 |
| | (water | | | |
| | based) | | | |
| Cetearyl | Thickner | 2 | 4 | 6 |
| Alcohol | (oil phase) | | | |
| (Lunette O) | | | | |
| and/or Cetyl | | | | |
| Alcohol | | | | |
| (Lanette 16) | | | | |
| Isopropyl | Stability | 2 | 2 | 2 |
| myristate | assistance | | | |
| | component | | | |
| TEA | Neutralizing | 0.25 | 0.25 | 0.25 |
| | Agent | | | |
| Euxyl K940 | Preservative | 0.25 | 0.25 | 0.25 |
| Steareth-21 | Emulsifier | 0.9 | 0.9 | 0.9 |
| (HLB = 15.55) | (solid) | | | |
| Glyceryl | Emulsifier | 0.1 | 0.1 | 0.1 |
| Sterarate | (solid) | | | |
| 1233zd | | 10 | 10 | 10 |
| 1234ze | | 40 | 40 | 40 |
| Total | | 100 | 100 | 100 |
| HLB of | | 14.38 | 14.38 | 14.38 |
| Emulsifiers in | | | | |
| Formulation | | | | |
| Stability | | Stable | Stable | Stable |
| Weight Ratio | | 0.94 | 1.88 | 2.82 |
| of Oil Phase | | | | |
| Thickeners to | | | | |
| Total Oil | | | | |
| Phase | | | | |
| Liquids | | | | |
| (exclusive of | | | | |
| 1233zd and | | | | |
| 1234ze) | | | | |
| Ratio Water | | 0.06 | 0.03 | 0.02 |
| Phase | | | | |
| Thickener to | | | | |
| Oil Phase | | | | |
| Thikener | | | | |
| Ratio of total | | 0.71 | 0.43 | 0.30 |
| oil phase | | | | |
| liquids to | | | | |
| total oil | | | | |
| phase solids | | | | |

As can be seen from the results reported above, all formulations were stable (after shaking for 24 hours at room temperature). However, even these stable formulations, when dispensed through the nozzle from the pressurized container formed a cooling foam, but not an ice cream foam. It is noted that formulations 2CB and 20C had a ratio by weight of oil phase thickeners to oil phase liquids (exclusive of 1233zd and 1234ze) greater than about 1.25 and less than 3, but the ratio of water phase thickener to oil phase thickener for all formulations that was 0.02 or greater.

### Example 21—Personal Care Formulation with High HLB Solid Emulsifier and Oil Phase Thickener—Stable Formulation; Ice Cream Foam not Formed

A personal care formulation as described in Table 21 below which contained 50% by weight of trans1233zd plus trans1234ze, and using a high HLB, solid non-ionic emulsifier were formed.

TABLE 21

| | | Wt % Component |
|---|---|---|
| Ingredient | Function | 21 |
| water | Solvent | 44 |
| Glycerin | humectant | 0.51 |
| Carbopol 904 | Thickener | 0.125 |
| | (water | |
| | based) | |
| Cetearyl Alcohol (Lanette O) | Thickner | 3 |
| and/or Cetyl Alcohol | (oil | |
| (Lanette 16) | phase) | |
| Ester oil (oil | Skin | 0 |
| phase liquid) | conditioning | |
| | agent | |
| Cyclopentasiloxane/ | Skin | 1.5 |
| Cyclopentasiloxane | conditioning | |
| (Xiameter PMX-0345) | agent and | |
| and Dimethicon | protectant | |
| Silicone liquid | | |
| TEA | Neutralizing | 0.25 |
| | Agent | |
| Euxyl K940 | Preservative | 0.25 |
| Steareth-21 | Emulsifier | 0.9 |
| (HLB = 15.55) | (solid) | |
| Glyceryl Sterarate | Emulsifier | 0.1 |
| | (solid) | |
| 1233zd | | 10 |
| 1234ze | | 40 |
| Total | | 100 |
| HLB of Emulsifiers | | 14.38 |
| in Formulation | | |
| Stability | | Stable |
| Weight Ratio of Oil Phase | | 1.85 |
| Thickeners to Total Oil | | |
| Phase Liquids (exclusive of | | |
| 1233zd and 1234ze) | | |
| Weight Ratio of Water phase | | 0.042 |
| thickeners to Oil Phase | | |
| Thickeners | | |

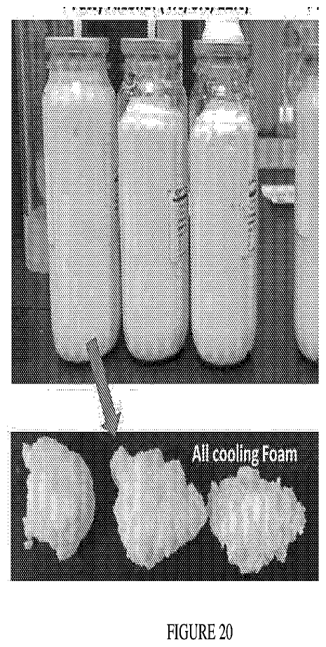

As can be seen from the results reported above, the formulation formed a stable emulsion (after shaking for 24 hours at room temperature), and when dispensed through the nozzle from the pressurized container formed a cooling foam as illustrated in FIG. 20. However, ice cream foam was not formed. It is noted that while Formulation 21 had weight ratios of oil phase thickeners to total oil phase liquids (exclusive of 1233zd and 1234ze) greater than 1.25 and less than about 3, the formulation had a weight ratio of water based thickener to oil based thickener that was well above 0.02.

### Comparative Example 1—Personal Care Formulation with Liquid, Low HLB Emulsifier—Formulation not Stable

A personal care formulation which contained 50% by weight of trans1233zd plus trans1234ze as depicted in Table C1 below is formed:

TABLE C1

| | | Wt % Component |
|---|---|---|
| Ingredient | Function | Formulation C1 |
| water | Solvent | 47.25 |
| Glycerin | humectant | 1 |
| Carbopol 940 | Thickener | 0.25 |
| | (water | |
| | based) | |
| TEA | Neutralizing | 0.25 |
| | Agent | |
| Euxyl K940 | Preservative | 0.25 |
| Brij 93 | Emulsifier | 1 |
| (HLB = 4) | (liquid) | |
| 1233zd | | 10 |
| 1234ze | | 40 |
| Total | | 100 |

As can be seen from the Table above, the HLB of the only emulsifier used in the formulation was Brij 93, which is a liquid emulsifier and provided the formulation with a net HLB value of about 4. A stable emulsion was not formed, as measured by whether phase separation is observed when shaken 24 hours after formation at room temperature. Unless otherwise indicated herein, the test described herein was used in all of the examples hereof to determine the stability of the formulation, with the observance of phase separation indicating that the formulation is not stable and will not be acceptable for forming a foam.

### Comparative Examples 2A, 2B, 2C, 2D and 2E—Unstable Personal Care Formulations

A series of personal care formulation generally as described in Comparative Example 1 and Examples 3A-3D which contained 50% by weight of trans1233zd plus trans1234ze, but using a water based thickener but without using an oil phase thickener, was prepared as indicated in Table as described in Table C2 below were formed:

TABLE C2

| | | Wt % Component | | | | |
|---|---|---|---|---|---|---|
| Ingredient | Function | C2A | C2B | C2C | C2D | C2E |
| water | Solvent | 47.5 | 47.5 | 47.5 | 47.5 | 47.5 |
| Glycerin | humectant | 1 | 1 | 1 | 1 | 1 |
| Carbopol 904 | Thickener | 0.25 | 0.25 | 0.25 | 0.25 | 0.25 |
| | (water based) | | | | | |
| TEA | Neutralizing | 0.25 | 0.25 | 0.25 | 0.25 | 0.25 |
| | Agent | | | | | |
| Euxyl K940 | Preservative | 0.25 | 0.25 | 0.25 | 0.25 | 0.25 |
| Steareth-21 | Emulsifier | 0.2 | 0.45 | 0.7 | 0.9 | 0.9 |
| (HLB = 15.55) | (solid) | | | | | |
| Glyceryl Sterarate | Emulsifier | 0.8 | 0.55 | 0.3 | 0.1 | 0 |
| | (solid) | | | | | |
| 1233zd | | 10 | 10 | 10 | 10 | 10 |
| 1234ze | | 40 | 40 | 40 | 40 | 40 |
| Total | | 100 | 100 | 100 | 100 | 100 |
| HLB of Emulsifiers | | 6.14 | 9.06 | 12 | 14.33 | 15.55 |
| in Formulation | | | | | | |

None of the formulation as described in Table C2 formed a stable emulsion.

### Comparative Example 3—Personal Care Formulation with Low HLB Emulsifier

A personal care formulation which contained 50% by weight of trans1233zd plus trans1234ze as depicted in Table C3 below is formed:

TABLE C3

| Ingredient | Function | Wt % Component |
|---|---|---|
| water | Solvent | 47.5 |
| Glycerin | humectant | 1 |
| Carbopol 904 | Thickener | 0.25 |
| | (water | |
| | based) | |
| TEA | Neutralizing | 0.25 |
| | Agent | |
| Euxyl K940 | Preservative | 0.25 |
| Brij 93 | Emulsifier | 1 |
| (HLB = 4) | (liquid) | |
| 1233zd | | 40 |
| 1234ze | | 10 |
| Total | | 100 |

As can be seen from the Table above, the HLB of the emulsifier used in the formulation was Brij 93, which provided the formulation with emulsifiers having an HLB value of about 4. A stable emulsion was not formed.

### Comparative Examples 4A, 4B, 4C and 4D—Unstable Personal Care Formulations

A series of personal care formulations were prepared using the components and the amounts as identified in Table C4 below by first forming a concentrate of the ingredients in the formulation (shown under the heading Conc. in Table 4C) and then preparing a full formulation by combining the concentrate in a 50:50 weight ratio with a combination of 1233zd and 1234ze, with this final formulation being identified under the headings “Final”:

TABLE C4

| | | | Wt % Component | | | | | | | |
|---|---|---|---|---|---|---|---|---|---|---|
| | | | C5A | | C5B | | C5C | | C5D | |
| Phase | Ingredient | Function | con. | Total | Con | Total | Con | Total | Con | Total |
| W | water | Solvent | 94.05 | 47.025 | 70.35 | 35.175 | 91.65 | 45.825 | 46.05 | 23.025 |
| W | EDTA-2Na | Water | 0.1 | 0.05 | 0.1 | 0.05 | 0.1 | 0.05 | 0.1 | 0.05 |
| | | stabilizer | | | | | | | | |
| W | AMP- | pH | 0.25 | 0.125 | 0.25 | 0.125 | 0.25 | 0.125 | 0.25 | 0.125 |
| | ULTRA | adjuster | | | | | | | | |
| | PC 2000 | | | | | | | | | |
| W | 1,2- | humectant | 5 | 2.5 | 5 | 2.5 | 5 | 2.5 | 5 | 2.5 |
| | Propylene | | | | | | | | | |
| | Glycol | | | | | | | | | |
| W | Natrosol ™ | Thickening | 0.1 | 0.05 | 0.1 | 0.05 | 0.1 | 0.05 | 0.1 | 0.05 |
| | 250HHR | agent | | | | | | | | |
| | Hydroxyet | | | | | | | | | |
| | hylcellulose | | | | | | | | | |
| | Total Water | | 99.5 | 49.75 | 75.8 | 37.9 | 97.1 | 48.55 | 51.5 | 25.75 |
| | Phase | | | | | | | | | |
| O | Pemulen | Synthetic | 0.2 | 0.1 | 0 | 0 | 0.2 | 0.1 | 0.2 | 0.1 |
| | TR-1 | Polymeric | | | | | | | | |
| | | emulsifier | | | | | | | | |
| O | Carbopol ® | Rheology | 0.2 | 0.1 | 0.2 | 0.1 | 0.2 | 0.1 | 0.2 | 0.1 |
| | Ultrez 10 | modifier | | | | | | | | |
| O | TEGOSOFT | emollient | 0 | 0 | 6 | 3 | 0.6 | 0.3 | 12 | 6 |
| | TN | and | | | | | | | | |
| | | texture | | | | | | | | |
| | | enhancing | | | | | | | | |
| | | agent | | | | | | | | |
| O | 2-EHP | emollient | 0 | 0 | 18 | 9 | 1.8 | 0.9 | 36 | 18 |
| O | Span 80 | Emulsifier | 0.1 | 0.05 | 0 | 0 | 0.1 | 0.05 | 0.1 | 0.05 |
| | (HLB 4.3) | (liquid) | | | | | | | | |
| | Total Oil (excluding | | 0.5 | 0.25 | 24.2 | 12.1 | 2.9 | 1.45 | 48.5 | 24.25 |
| | zd and zd) | | | | | | | | | |
| O | 1233zd | | | 40 | | 40 | | 40 | | 40 |
| O | 1234ze | | | 10 | | 10 | | 10 | | 10 |
| O | Total Oil (incl | | | 50.25 | | 62.1 | | 51.45 | | 74.25 |
| | zdandzd) | | | | | | | | | |
| | Total Oil + | | 100 | 100 | 100 | 100 | 100 | 100 | 100 | 100 |
| | Water Phase | | | | | | | | | |
| | HLB of Emulsifiers | | 4.3 | | 4.3 | 4.3 | 4.3 | | 4.3 | 4.3 |
| | in Formulation | | | | | | | | | |
| WEIGHT RATIOS | | | | | | | | | | |
| | 1233ZDN2 34ZE | | 4.00 | | | 4.00 | | 4.00 | | 4.00 |
| | Oil:Water | | 0.01 | | 0.32 | | 0.03 | | 0.94 | |
| | Concenttrate | | | | | | | | | |
| | Total Oil:polymeric | | | 503 | | NA | | 515 | | 743 |
| | emulsifiier wt ratio | | | | | | | | | |
| | in final | | | | | | | | | |
| | Total Oil:Total | | | 335 | | NA | | 343 | | 495 |
| | Emulsifier wt ratio | | | | | | | | | |
| | in final | | | | | | | | | |
| | Oil:Water Total | | | 1.00 | | 1.64 | | 1.06 | 0 | 2.88 |
| PROPERTIES | | | | | | | | | | |
| | Phase Stability | | | No | | No | | No | | No |
| | (Yes or No) | | | | | | | | | |
| | Bubbling Foam (Yes | | | NA | | NA | | NA | | NA |
| | or No) | | | | | | | | | |

As can be seen from Table C4 above, the personal care formulation C4B included neither an emulsifier with an HLB above 5 nor an emulsification polymer, such as Pemulen TR-1, and this formulation did not form a stable emulsion. While Formulations C4A and C4C did include the emulsification polymer Pemulen TR-1, it was not present in an amount effective to provide a stable emulsion, that is, this formulation had a weight ratio of total oil:Pemulen TR-1 of only 503 and 515, respectfully, which for this formulation were not sufficient to form a stable emulsion. Formulation C4D also failed to form a stable emulsion even though it included the emulsification polymer Pemulen TR-1. That is, the Pemulen was not present in an amount effective to provide a stable emulsion since in this formulation the weight ratio of total oil:Pemulen TR-1 was 743, which for this formulation were not sufficient to form a stable emulsion.

### Comparative Example C5A—Failure of Ultra Crackling Foam—Reduced Liquid Emulsifier and Reduced Film Former/Solid Emulsifier

A personal care formulation C5A was formed with concentrations of trans1234ze greater than 60% by weight and with trans11233zd:trans11234ze in a weight ratio of 7.56:1, but with reduced levels of liquid emulsifier and film former/solid emulsifier, as depicted in Table C5A below were formed. These formulations were sprayed from a container as described herein to form a foam. The results are reported in Table C5 below.

TABLE C5A

| | | Wt % Component |
|---|---|---|
| Ingredient | Function | C5A |
| water | Solvent | 17.825 |
| Forlan C24 (HLB 13) | Film forming agent? | 0.161 |
| polyethylene glycol | Solid Emulsifier? | |
| ether of cholesterol | | |
| and a polyethylene | | |
| glycol ether of cetyl | | |
| alcohol - light solid | | |
| flake | | |
| Natrosol ™ 250 HR | Thickner (water | 0.368 |
| (Hydroxyethylcellulose) | based) | |
| Tween-20 (HLB = 16.7) | Emulsifier (non-ionic | 0.046 |
| Polyethylene glycol sorbitan | viscous liquid) | |
| monolaurate, | | |
| (polyoxyethylene (20)) | | |
| Ethanol (200 Proof) | ?_{——}? | 4.6 |
| 1233zd | | 68 |
| 1234ze | | 9 |
| Total | | 100 |
| RATIOS (weight) | | |
| 1233zd:1234ze | | 7.56 |
| 1233zd:ethanol | | 14.8 |
| 1233zd:liquid | | 1478 |
| emulsifier | | |
| 1233zd: film former | | 422 |
| 1233zd:thickner | | 184 |
| Liquid | | 0.125 |
| Emulsifier:thickner | | |
| TEST RESULTS | | |
| Spray Quality* | | 5 |
| Foam Quality** | | 5 |
| Phase stability | | Stable |
| Cracking | | 3 |
| Loudness*** | | |

*1-5 range (1 = hard to spray, 5 = easily sprayed)
**1-5 range (1 = flat foam (poor quality), 5 = good quality foam)
***1-5 range (1 = low sound, 5 = loud (ultra) crackling)

As can be seen from the table above, formulation C5A had a reduced level of liquid emulsifier (1234z4:liquid emulsifier ratio=1478) as well as a reduced level of film forming agent. While this formulation was stable and otherwise acceptable, it did not produce a loudness value of 5 and therefore is not considered ultra crackling foam.

### Comparative Examples C51B and C5C—Failure of Ultra Crackling Foam-Thickener Levels

Two personal care formulations (C5B and C5C) are formed with concentrations of trans11234ze greater than 60% by weight and with trans1233zd:trans1234ze in a weight ratio of 7.56:1, but with higher and lower levels of thickening agent, as depicted in Table C5B3 below and sprayed from a container as described herein to form a foam. The results are reported in Table C5B3 below.

TABLE C5B

| | | Wt % Component | |
|---|---|---|---|
| Ingredient | Function | C5A | C5B |
| water | Solvent | 17.825 | 17.338 |
| Forlan C24 (HLB 13) | Solid Emulsifier | 0.161 | 0.644 |
| polyethylene glycol | | | |
| ether of cholesterol | | | |
| and a polyethylene | | | |
| glycol ether of cetyl | | | |
| alcohol - light solid | | | |
| flake | | | |
| Natrosol ™ 250 HR | Thickener (water | 0.368 | 0.184 |
| (Hydroxyethylcellulose) | based) | | |
| Tween-20 (HLB = 16.7) | Emulsifier (non- | 0.046 | 0.184 |
| Polyethylene glycol | ionic viscous liquid) | | |
| sorbitan monolaurate, | | | |
| (polyoxyethylene (20)) | | | |
| Ethanol (200 Proof) | | 4.6 | 4.6 |
| 1233zd | | 68 | 68 |
| 1234ze | | 9 | 9 |
| Total | | 100 | 100 |
| RATIOS (weight) | | | |
| 1233zd:1234ze | | 7.56 | 7.56 |
| 1233zd:ethanol | | 14.8 | 14.8 |
| 1233zd:liquid | | 1478 | 370:1 |
| emulsifier | | | |
| 1233zd:film former | | 422 | 105.6:1 |
| 1233zd:thickner | | 184 | 370:1 |
| Liquid | | 0.125 | |
| Emulsifier:thickner | | | |
| TEST RESULTS | | | |
| Spray Quality* | | 5 | 5 |
| Foam Quality** | | 5 | 5 |
| Phase stability | | Stable | Stable |
| Cracking | | 3 | 2 |
| Loudness*** | | | |

*1-5 range (1 = hard to spray, 5 = easily sprayed)
**1-5 range (1 = flat foam (poor quality), 5 = good quality foam)
***1-5 range (1 = low sound, 5 = loud (ultra) crackling)

As can be seen from the table above, formulation C5B had a reduced level of thickener (1233zd:thickener ratio=370). While this formulation was stable and otherwise acceptable, it did not produce a loudness value of 5 and therefore is not considered ultra crackling foam.

As can be seen from the table above, formulation C5C had an increased level of thickener (1233zd:thickener ratio=123). While this formulation was stable and produced a loudness value of 5, it was not an acceptable formulation because it had unacceptable spray characteristics and because the foam characteristics were poor.

### Comparative Examples C5D and C5E—Failure of Ultra Crackling Foam—Only Liquid and Only Solid Emulsifier

Two personal care formulations (C5D and C5E are formed with concentrations of trans1234ze greater than 60% by weight and with trans1233zd:trans1234ze in a weight ratio of 7.56:1, but with the use of no liquid emulsifier in one case and no film former/solid emulsifier in the other as depicted in Table C5D below, and sprayed from a container as described herein to form a foam. The results are reported in Table C5D below.

TABLE 14

| | | Wt % Component | |
|---|---|---|---|
| Ingredient | Function | C5D | C5E |
| water | Solvent | 17.204 | 17.204 |
| Forlan C24 (HLB 13) | Solid | 0.828 | 0 |
| polyethylene glycol | Emulsifier | | |
| ether of cholesterol | | | |
| and a polyethylene | | | |
| glycol ether of cetyl | | | |
| alcohol - light solid | | | |
| flake | | | |
| Natrosol ™ 250 HR | Thickner | 0.368 | 0.368 |
| (Hydroxyethylcellulose) | (water based) | | |
| Tween-20 (HLB = 16.7) | Emulsifier | 0 | 0.828 |
| Polyethylene glycol | (non-ionic | | |
| sorbitan monolaurate, | viscous | | |
| (polyoxyethylene 20)) | liquid) | | |
| Ethanol (200 Proof) | | 4.6 | 4.6 |
| 1233zd | | 68 | 68 |
| 1234ze | | 9 | 9 |
| Total | | 100 | 100 |
| RATIOS (weight) | | | |
| 1233zd:1234ze | | 7.56 | 7.56 |
| 1233zd:ethanol | | 14.8 | 14.8 |
| 1233zd:liquid | | NA | 82.5:1 |
| emulsifier | | | |
| 1233zd:film | | 82.5:1 | NA |
| former | | | |
| 1233zd:thickner | | 184 | 184:1 |
| Liquid | | NA | 2.25 |
| Emulsifier:thickner | | | |
| TEST RESULTS | | | |
| Spray Quality* | | 5 | 5 |
| Foam Quality** | | 5 | 5 |
| Phase stability | | Stable | Separated |
| | | | over time |
| Cracking | | 1 | 5 |
| Loudness*** | | | |

*1-5 range (1 = hard to spray, 5 = easily sprayed)
**1-5 range (1 = flat foam (poor quality), 5 = good quality foam)
***1-5 range (1 = low sound, 5 = loud (ultra) crackling)

As can be seen from the table above, formulation C5D had no liquid emulsifier and C5E had not film former/solid emulsifier. While the formulation with no liquid emulsifier was stable and otherwise acceptable as a foam, it did not produce an ultra crackling effect. On the other hand, the formulation with no film former/solid emulsifier produced in an acceptable foam with an ultra crackling effect, it was not stable.

What is claimed is:

1. A method of treating human skin, hair and/or mucous membranes comprising:
(a) providing a personal care composition in a container under pressure in the form of a stable oil-in-water emulsion comprising:
(i) a continuous aqueous phase comprising water;
(ii) a discontinuous oil phase comprising trans-1-chloro-3,3,3-trifluoropropene (hereinafter 1233zd(E)) and/or trans-1-3,3,3-tetrafluoropropene (hereinafter 1234ze(E)), wherein said 1233zd(E) and said 1234ze (E) when together comprise at least about 40% by weight of said personal care composition and are present in a trans1233zd:trans1234ze weight ratio of from about 1:18 to less than about 8:1; and
(iii) an emulsifier, which may be present in the continuous and/or discontinuous phase, comprising (a) a non-ionic emulsifier having an HLB of from about 7 to about 19; and/or (b) a polymeric emulsifier; (c) and optionally a cationic emulsifier, said emulsifier (iii) being present in an amount effective to ensure that said oil-in-water emulsion is stable, and;
v) one or more active ingredients which may be present in the continuous and/or discontinuous phase; and
(vi) optionally one or more auxiliary gel, lotion or cream forming components which may be present in the continuous and/or discontinuous phase; and
(b) dispensing said personal care composition on and/or into the skin, hair and/or mucous membrane of the human being treated by discharging at least a portion of the personal care composition from said container into ambient pressure conditions to form a material on the user in a form selected from sherbet foam; ice cream foam; crackling foam; ultra crackling foam, bubbling foam; clear foam, gel and/or lotion; or a combination of two or more of these, with the provisos that:
(1) for said sherbet foam and said crackling foam the weight ratio of said 1233zd to said 1234ze is from 8:1 to about 1:1.5, said emulsifier comprises said non-ionic emulsifier and said personal care composition comprises a thickener for said continuous and/or said discontinuous phase;
(2) for said ice cream foam (i) the weight ratio of said 1233zd to said 1234ze is from 1:1.5 to about 1:8; (ii) said emulsifier comprises a solid non-ionic emulsifier having an HLB of from about 7 to about 198 and wherein the net HLB of the emulsifiers in formulation is from about 7 to 19; (iii) said personal care composition comprises a solid oil phase thickener and optionally an aqueous phase thickener, provided that the weight ratio of the continuous phase thickener to the discontinuous phase thickener is equal to or less than 0.02, or less than about 0.015, or less than 0.01, or less than 0.005; (iv) and said personal care composition comprises at least about 3% by weight of oil phase liquids, wherein the weight ratio of said oil phase thickeners to said total oil phase liquids is greater than 1.25 and less than about 3 and;
(3) for said ultra crackling foam, the personal care composition comprises at least about 60% by weight of said 1234ze, said ratio of said 1233zd to said 1234ze is from about 1:4 to about 1:18, said emulsifier comprises said non-ionic liquid emulsifier, and greater than 3% by weight and less than about 7% by weight based on all to components in the personal care composition of one or more polar carriers having a normal boiling point or an initial boiling point of from about 60° C. to about 200° C.;
(4) for bubbling foam, said emulsifier comprises said polymeric emulsifier in a weight ratio of total oil components to polymeric emulsifier of from about greater than about 515 and less than about 740; and
(5) for clear foam, gel and/or lotion, said discontinuous phase comprises a discontinuous phase refractive index balancer (DRIB) in an amount effective to produce a weight ratio of said 1233zd(E):DRIB of from about 3:1 to about 30:1.

2. The method of claim 1 wherein the method forms ice cream foam.

3. Ice cream foam formed by the method of claim 2.

4. The method of claim 1 wherein the method forms sherbet foam.

5. Sherbet foam formed by the method of claim 4.

6. The method of claim 1 wherein the method forms bubbling foam.

7. Bubbling foam formed by the method of claim 7.

8. The method of claim 1 wherein the method forms ultra-crackling foam.

9. Ultra-crackling foam formed by the method of claim 8.

10. The method of claim 1 wherein the method forms a clear foam, gel or lotion.

11. A clear foam, gel or lotion foam formed by the method of claim 10.

12. The method of claim 10 wherein said DRIB has a refractive index of from about 1.375 to about 1.7.

13. The method of claim 10 wherein said aqueous phase further comprises one or more continuous phase refractive index balancers (CRIB) in an amount effective to produce a refractive index of the continuous phase of from about 1.34 to about 1.37.

14. The method of claim 10 wherein said DRIB is present in an amount effective to produce a refractive index of the discontinuous phase equal to the refractive index of the continuous phase+/−0.005, wherein the weight ratio of said 1233zd(E):DRIB is from about 3:1 to about 15:1.

15. The method of claim 13 wherein said DRIB is present in an amount effective to produce a refractive index of the discontinuous phase equal to the refractive index of the continuous phase+/−0.005, wherein the weight ratio of said 1233zd(E):DRIB is from about 3:1 to about 15:1.

16. The method of claim 13 wherein said 1233zd(E) is present in the oil-in-water emulsion in an amount of 20% by weight or less based on all the components in the oil-in-water emulsion.

17. The method of claim 10 wherein said personal care composition comprises an emulsifier having an HLB value of from about 7 to about 18 and which may be present in the continuous and/or discontinuous phase.

18. The method of claim 17 wherein said emulsifier comprising a copolymer comprising an acrylate moiety and/or a taurate moiety.

19. A personal care formulation for treating human skin, hair and/or mucous membranes to form an ice cream foam comprising a stable oil-in-water emulsion comprising:
(i) a continuous aqueous phase comprising water;
(ii) a discontinuous oil phase comprising 1233zd(E)) and 1234ze(E), wherein said 1233zd(E) and said 1234ze(E) together comprise at least about 40% by weight of said personal care composition and are present in a trans1233zd:trans1234ze weight ratio of from about 1:1.5 to 1:8; and
(iii) an emulsifier, which may be present in the continuous and/or discontinuous phase, comprising (a) one or more solid non-ionic emulsifiers having an HLB of from about 7 to about 19 wherein the net HLB of the formulation is from about 7 to about 19; (b) optionally a polymeric emulsifier, and (c) optionally a cationic emulsifier, said emulsifier (iii) being present in an amount effective to ensure that said oil-in-water emulsion is stable;
(iv) a solid thickener for said discontinuous oil phase, and optionally an aqueous phase thickener, provided that the weight ratio of the aqueous phase thickener to the oil phase thickener is equal to or less than 0.02;
(v) at least about 3% by weight of oil phase liquids, wherein the weight ratio of said oil phase thickeners to said total oil phase liquids is greater than 1.25 and less than about 3; and
(vi) optionally one or more auxiliary gel, lotion or cream forming components which may be present in the continuous and/or discontinuous phase.

20. A personal care formulation for treating human skin, hair and/or mucous membranes to form an sherbet foam in the form of a stable oil-in-water emulsion comprising:
(i) a continuous aqueous phase comprising water;
(ii) a discontinuous oil phase comprising 1233zd(E) and 1234ze(E), wherein said 1233zd(E) and said 1234ze (E) together comprise at least about 40% by weight of said personal care composition and are present in a trans1233zd:trans1234ze weight ratio of from about 8:1 to about 1:1.5; and
(iii) one or more emulsifiers, which may be present in the continuous and/or discontinuous phase, having a net HLB of from about 7 to about 19 and comprising at least one non-ionic emulsifier; (b) optionally a polymeric emulsifier; and (c) optionally a cationic emulsifier, said emulsifier (iii) being present in an amount effective to ensure that said oil-in-water emulsion is stable, and;
(iv) a thickener for said continuous and/or said discontinuous phase;
(iv) one or more active ingredients which may be present in the continuous and/or discontinuous phase; and
(v) optionally one or more auxiliary gel, lotion or cream forming components which may be present in the continuous and/or discontinuous phase.
